(19) United States
(12) Patent Application Publication
(10) Pub. No.: US 2023/0042653 A1
(43) Pub. Date: Feb. 9, 2023

(54) COMBINATIONS

(71) Applicant: Recurium IP Holdings, LLC, San Diego, CA (US)

(72) Inventors: Ahmed Abdi Samatar, San Diego, CA (US); Jianhui Ma, San Diego, CA (US); Jiali Li, San Diego, CA (US); Peter Qinhua Huang, San Diego, CA (US); Sayee Gajanan Hegde, San Diego, CA (US); Kevin Duane Bunker, Escondido, CA (US); Fernando Donate, Santa Monica, CA (US)

(21) Appl. No.: 17/757,511

(22) PCT Filed: Dec. 16, 2020
(86) PCT No.: PCT/US2020/065411

§ 371 Date: Jun. 16, 2022

Related U.S. Application Data
(60) Provisional application No. 63/009,754, filed on Apr. 14, 2020.
(60) Provisional application No. 62/952,042, filed on Dec. 20, 2019.

(51) Int. Cl. A61K 31/437; A61K 31/519; A61P 35/00

(52) U.S. Cl. CPC A61K 31/437; A61K 31/519; A61P 35/00

(57) ABSTRACT

Disclosed herein are combinations of compounds for treating a disease or condition, such as cancer. A combination of compounds for treating a disease or condition can include a SERD inhibitor and a WEE1 inhibitor, along with pharmaceutically acceptable salts of any of the foregoing.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6, including U.S. Provisional Application Nos. 62/952,042, filed Dec. 20, 2019 and 63/009,754, filed Apr. 14, 2020.

### FIELD

The present application relates to the fields of chemistry, biochemistry and medicine. More particularly, disclosed herein are combination therapies, and methods of treating diseases and/or conditions with a combination therapies descried herein.

### DESCRIPTION

Cancers are a family of diseases that involve abnormal cell growth with the potential to invade or spread to other parts of the body. Cancer treatments today include surgery, hormone therapy, radiation, chemotherapy, immunotherapy, targeted therapy and combinations thereof. Survival rates vary by cancer type and by the stage at which the cancer is diagnosed. In 2019, roughly 1.8 million people will be diagnosed with cancer, and an estimated 606,880 people will die of cancer in the United States. Thus, there still exists a need for effective cancer treatments.

### SUMMARY

Some embodiments described herein relate to a combination of compounds that can include an effective amount of Compound (A), or a pharmaceutically acceptable salt thereof, and an effective amount of one or more of Compound (B), or a pharmaceutically acceptable salt thereof.

Other embodiments described herein relate to a combination of compounds that can include an effective amount of Compound (C), or a pharmaceutically acceptable salt thereof, and an effective amount of one or more of Compound (B), or a pharmaceutically acceptable salt thereof.

Some embodiments described herein relate to the use of a combination of compounds for treating a disease or condition, wherein the combination includes an effective amount of Compound (A), or a pharmaceutically acceptable salt thereof, and an effective amount of one or more of Compound (B), or a pharmaceutically acceptable salt thereof. Other embodiments described herein relate to the use of a combination of compounds in the manufacture of a medicament for treating a disease or condition, wherein the combination includes an effective amount of Compound (A), or a pharmaceutically acceptable salt thereof, and an effective amount of one or more of Compound (B), or a pharmaceutically acceptable salt thereof.

Some embodiments described herein relate to the use of a combination of compounds for treating a disease or condition, wherein the combination includes an effective amount of Compound (C), or a pharmaceutically acceptable salt thereof, and an effective amount of one or more of Compound (B), or a pharmaceutically acceptable salt thereof. Other embodiments described herein relate to the use of a combination of compounds in the manufacture of a medicament for treating a disease or condition, wherein the combination includes an effective amount of Compound (C), or a pharmaceutically acceptable salt thereof, and an effective amount of one or more of Compound (B), or a pharmaceutically acceptable salt thereof.

In some embodiments, the disease or condition can be a cancer described herein.

### DRAWINGS

FIG. 1 provides examples of WEE1 inhibitors.

FIG. 2 shows the percent inhibition of single agent and combination treatments of Compound (A) and Compound 1 against MCF-7 breast cancer cells.

FIG. 3 shows the results of a combination study of Compound (A) with Compound 1 in a MCF-7 xenograft tumor model.

### DETAILED DESCRIPTION

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, cycloalkynyl, aryl, heteroaryl, heterocyclyl, aryl(alkyl), cycloalkyl(alkyl), heteroaryl(alkyl), heterocyclyl(alkyl), hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, nitro, sulfenyl, sulfinyl, sulfonyl, haloalkyl, haloalkoxy, an amino, a mono-substituted amino group and a di-substituted amino group.

As used herein, “Cₐ to C_{b}” in which “a” and “b” are integers refer to the number of carbon atoms in a group. The indicated group can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated, the broadest range described in these definitions is to be assumed.

If two “R” groups are described as being “taken together” the R groups and the atoms they are attached to can form a cycloalkyl, cycloalkenyl, aryl, heteroaryl or heterocycle. For example, without limitation, if R^{a} and R^{b} of an NR^{a}R^{b} group are indicated to be “taken together,” it means that they are covalently bonded to one another to form a ring:

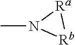

As used herein, the term “alkyl” refers to a fully saturated aliphatic hydrocarbon group. The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkyl” where no numerical range is designated). The alkyl group may also be a medium size alkyl having 1 to 12 carbon atoms. The alkyl group could also be a lower alkyl having 1 to 6 carbon atoms. An alkyl group may be substituted or unsubstituted.

The term “alkenyl” used herein refers to a monovalent straight or branched chain radical of from two to twenty carbon atoms containing a carbon double bond(s) including, but not limited to, 1-propenyl, 2-propenyl, 2-methyl-1-propenyl, 1-butenyl, 2-butenyl and the like. An alkenyl group may be unsubstituted or substituted.

The term “alkynyl” used herein refers to a monovalent straight or branched chain radical of from two to twenty carbon atoms containing a carbon triple bond(s) including, but not limited to, 1-propynyl, 1-butynyl, 2-butynyl and the like. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused, bridged or spiro fashion. As used herein, the term “fused” refers to two rings which have two atoms and one bond in common. As used herein, the term “bridged cycloalkyl” refers to compounds wherein the cycloalkyl contains a linkage of one or more atoms connecting non-adjacent atoms. As used herein, the term “spiro” refers to two rings which have one atom in common and the two rings are not linked by a bridge. Cycloalkyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). A cycloalkyl group may be unsubstituted or substituted. Typical mono-cycloalkyl groups include, but are in no way limited to, cyclopropyl, cyclobutyl, cyclopentyl, cyclohexyl, cycloheptyl, and cyclooctyl. Examples of fused cycloalkyl groups are decahydronaphthalenyl, dodecahydro-1H-phenalenyl and tetradecahydroanthracenyl; examples of bridged cycloalkyl groups are bicyclo[1.1.1]pentyl, adamantanyl, and norbornanyl; and examples of spiro cycloalkyl groups include spiro[3.3]heptane and spiro[4.5]decane.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). Cycloalkenyl groups can contain 3 to 10 atoms in the ring(s) or 3 to 8 atoms in the ring(s). When composed of two or more rings, the rings may be connected together in a fused, bridged or spiro fashion. A cycloalkenyl group may be unsubstituted or substituted.

As used herein, “cycloalkynyl” refers to a mono- or multi-cyclic hydrocarbon ring system that contains one or more triple bonds in at least one ring. If there is more than one triple bond, the triple bonds cannot form a fully delocalized pi-electron system throughout all the rings. Cycloalkynyl groups can contain 6 to 10 atoms in the ring(s) or 6 to 8 atoms in the ring(s). When composed of two or more rings, the rings may be joined together in a fused, bridged or spiro fashion. A cycloalkynyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted.

As used herein, “heteroaryl” refers to a monocyclic or multicyclic aromatic ring system (a ring system with fully delocalized pi-electron system) that contain(s) one or more heteroatoms (for example, 1, 2 or 3 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” or “heteroalicyclyl” refers to three-, four-, five-, six-, seven-, eight-, nine-, ten-, up to 18-membered monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused, bridged or spiro fashion. As used herein, the term “fused” refers to two rings which have two atoms and one bond in common. As used herein, the term “bridged heterocyclyl” or “bridged heteroalicyclyl” refers to compounds wherein the heterocyclyl or heteroalicyclyl contains a linkage of one or more atoms connecting non-adjacent atoms. As used herein, the term “spiro” refers to two rings which have one atom in common and the two rings are not linked by a bridge. Heterocyclyl and heteroalicyclyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). Additionally, any nitrogens in a heteroalicyclic may be quaternized. Heterocyclyl or heteroalicyclic groups may be unsubstituted or substituted. Examples of such “heterocyclyl” or “heteroalicyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, azepane, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and/or 3,4-methylenedioxyphenyl). Examples of spiro heterocyclyl groups include 2-azaspiro[3.3]heptane, 2-oxaspiro[3.3]heptane, 2-oxa-6-azaspiro[3.3]heptane, 2,6-diazaspiro[3.3]heptane, 2-oxaspiro[3.4]octane and 2-azaspiro[3.4]octane.

As used herein, “aralkyl” and “aryl(alkyl)” refer to an aryl group connected, as a substituent, via a lower alkylene group. The lower alkylene and aryl group of an aralkyl may be substituted or unsubstituted. Examples include but are not limited to benzyl, 2-phenylalkyl, 3-phenylalkyl and naphthylalkyl.

As used herein, “heteroaralkyl” and “heteroaryl(alkyl)” refer to a heteroaryl group connected, as a substituent, via a lower alkylene group. The lower alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl and imidazolylalkyl and their benzo-fused analogs.

A “heteroalicyclyl(alkyl)” and “heterocyclyl(alkyl)” refer to a heterocyclic or a heteroalicyclylic group connected, as a substituent, via a lower alkylene group. The lower alkylene and heterocyclyl of a (heteroalicyclyl)alkyl may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl) and 1,3-thiazinan-4-yl(methyl).

As used herein, “lower alkylene groups” are straight-chained —CH₂— tethering groups, forming bonds to connect molecular fragments via their terminal carbon atoms. Examples include but are not limited to methylene (—CH₂—), ethylene (—CH₂CH₂—), propylene (—CH₂CH₂CH₂—) and butylene (—CH₂CH₂CH₂CH₂—). A lower alkylene group can be substituted by replacing one or more hydrogen of the lower alkylene group and/or by substituting both hydrogens on the same carbon with a cycloalkyl group (e.g.,

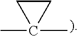

As used herein, the term “hydroxy” refers to a —OH group.

As used herein, “alkoxy” refers to the Formula —OR wherein R is an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) is defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

As used herein, “acyl” refers to a hydrogen, alkyl, alkenyl, alkynyl, aryl, heteroaryl, heterocyclyl, aryl(alkyl), heteroaryl(alkyl) and heterocyclyl(alkyl) connected, as substituents, via a carbonyl group. Examples include formyl, acetyl, propanoyl, benzoyl and acryl. An acyl may be substituted or unsubstituted.

A “cyano” group refers to a “—CN” group.

The term “halogen atom” or “halogen” as used herein, means any one of the radio-stable atoms of column 7 of the Periodic Table of the Elements, such as, fluorine, chlorine, bromine and iodine.

A “thiocarbonyl” group refers to a “—C(═S)R” group in which R can be the same as defined with respect to O-carboxy. A thiocarbonyl may be substituted or unsubstituted.

An “O-carbamyl” group refers to a “—OC(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-carbamyl may be substituted or unsubstituted.

An “N-carbamyl” group refers to an “ROC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-carbamyl may be substituted or unsubstituted.

An “O-thiocarbamyl” group refers to a “—OC(═S)—N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-thiocarbamyl may be substituted or unsubstituted.

An “N-thiocarbamyl” group refers to an “ROC(═S)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A C-amido may be substituted or unsubstituted.

An “N-amido” group refers to a “RC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-amido may be substituted or unsubstituted.

An “S-sulfonamido” group refers to a “—SO₂N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An S-sulfonamido may be substituted or unsubstituted.

An “N-sulfonamido” group refers to a “RSO₂N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-sulfonamido may be substituted or unsubstituted.

An “O-carboxy” group refers to a “RC(═O)O—” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “nitro” group refers to an “—NO₂” group.

A “sulfenyl” group refers to an “—SR” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

As used herein, “haloalkyl” refers to an alkyl group in which one or more of the hydrogen atoms are replaced by a halogen (e.g., mono-haloalkyl, di-haloalkyl and tri-haloalkyl). Such groups include but are not limited to, chloromethyl, fluoromethyl, difluoromethyl, trifluoromethyl, 1-chloro-2-fluoromethyl and 2-fluoroisobutyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy and 2-fluoroisobutoxy. A haloalkoxy may be substituted or unsubstituted.

The term “amino” as used herein refers to a —NH2 group.

A “mono-substituted amino” group refers to a “—NHR” group in which R can be an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. A mono-substituted amino may be substituted or unsubstituted. Examples of mono-substituted amino groups include, but are not limited to, —NH(methyl), —NH(phenyl) and the like.

A “di-substituted amino” group refers to a “—NR_{A}R_{B}” group in which R_{A} and R_{B} can be independently an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. A di-substituted amino may be substituted or unsubstituted. Examples of di-substituted amino groups include, but are not limited to, —N(methyl)₂, —N(phenyl)(methyl), —N(ethyl)(methyl) and the like.

Where the numbers of substituents is not specified (e.g. haloalkyl), there may be one or more substituents present. For example “haloalkyl” may include one or more of the same or different halogens. As another example, “C₁-C₃ alkoxyphenyl” may include one or more of the same or different alkoxy groups containing one, two or three atoms.

As used herein, a radical indicates species with a single, unpaired electron such that the species containing the radical can be covalently bonded to another species. Hence, in this context, a radical is not necessarily a free radical. Rather, a radical indicates a specific portion of a larger molecule. The term “radical” can be used interchangeably with the term “group.”

As used herein, when a chemical group or unit includes an asterisk (*), that asterisk indicates a point of attachment of the group or unit to another structure.

As used herein, “linking groups” are chemical groups that are indicated as having multiple open valencies for connecting to two or more other groups. For example, lower alkylene groups of the general formula —(CH₂)ₙ— where n is in the range of 1 to 10, are examples of linking groups that are described elsewhere herein as connecting molecular fragments via their terminal carbon atoms. Other examples of linking groups include —(CH₂)ₙO—, —(CH₂)ₙNH—, —(CH₂)ₙN(C₁-C₆alkyl)-, and —(CH₂)ₙS—, wherein each n is 0, 1, 2, 3, 4, 5, 6, 7, 8, 9, or 10. Those skilled in the art will recognize that n can be zero for some linking groups such as —(CH₂)ₙO—, in which case the linking group is simply —O—. Those skilled in the art will also recognize that reference herein to an asymmetrical linking group will be understood as a reference to all orientations of that group (unless stated otherwise). For example, reference herein to —(CH₂)ₙO— will be understood as a reference to both —(CH₂)ₙO— and —O—(CH₂)ₙ—.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), a sulfuric acid, a nitric acid and a phosphoric acid (such as 2,3-dihydroxypropyl dihydrogen phosphate). Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, trifluoroacetic, benzoic, salicylic, 2-oxopentanedioic, or naphthalenesulfonic acid. Pharmaceutical salts can also be obtained by reacting a compound with a base to form a salt such as an ammonium salt, an alkali metal salt, such as a sodium, a potassium or a lithium salt, an alkaline earth metal salt, such as a calcium or a magnesium salt, a salt of a carbonate, a salt of a bicarbonate, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine. For compounds of Formulae (A), (B) and (C), those skilled in the art understand that when a salt is formed by protonation of a nitrogen-based group (for example, NH₂), the nitrogen-based group can be associated with a positive charge (for example, NH₂ can become NH₃⁺) and the positive charge can be balanced by a negatively charged counterion (such as Cl⁻).

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Compounds

Some embodiments disclosed herein relate to the use of a combination of compounds for treating a disease or condition, wherein the combination can include an effective amount of Compound (A), or a pharmaceutically acceptable salt thereof, and an effective amount of one or more of Compound (B), or a pharmaceutically acceptable salt thereof, wherein: the Compound (A) has the structure:

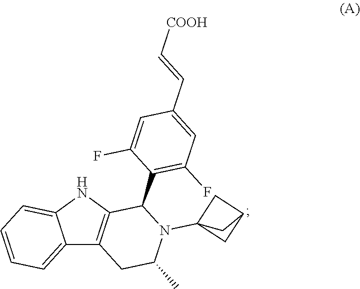 [SMILES: C[C@@H]1Cc2c([nH]c3ccccc23)[C@@H](c2c(F)cc(/C=C/C(=O)O)cc2F)N1C12CC(C1)C2]

and the one or more of Compound (B) can be a WEE1 inhibitor, or a pharmaceutically acceptable salt thereof, wherein the WEE1 inhibitor can be selected from AZD 1775, NUV-569, IMP7068, Debio 0123, SC0191 and PD-166285, or a pharmaceutically acceptable salt of any of the foregoing

Compound (A) can be a salt. For example, in some embodiments, Compound (A) can be a hydrogen sulfate salt. Those skilled in the art understand that the hydrosulfate salt of Compound (A) has a single molecule of Compound (A) for a single molecule of hydrogen sulfate. In other embodiments, Compound (A) can be a sulfate salt. Those skilled in the art understand that the sulfate salt of Compound (A) has two molecules of Compound (A) for a single molecule of sulfate. Further, those skilled in the art understand that hydrogen sulfate and sulfate salts of Compound (A) are where the nitrogen of Compound (A) can be protonated.

In some embodiments, Compound (A) can be a pharmaceutically acceptable salt form of Compound (A) that can include the hydrosulfate salt of Compound A and the sulfate salt of Compound (A). As an example, a pharmaceutically acceptable salt form of Compound (A) can be a pharmaceutically acceptable salt form of Compound (A) that consists essentially of the hydrosulfate salt of Compound (A) and the sulfate salt of Compound (A). Exemplary salt forms of Compound (A) include Form A and Form C. In some embodiments, Compound (A), or a pharmaceutically acceptable salt thereof, can be Form A. In some embodiments, Compound (A), or a pharmaceutically acceptable salt thereof, can be Form C. In some embodiments, Compound (A), or a pharmaceutically acceptable salt thereof, can include Form A and Form C. Additional details regarding Form A and Form C of Compound (A) are provided in International Application No. PCT/US2020/058526, filed Nov. 2, 2020, which is hereby incorporated by reference in its entirety.

Other embodiments disclosed herein relate to the use of a combination of compounds for treating a disease or condition, wherein the combination can include an effective amount of Compound (C), or a pharmaceutically acceptable salt thereof, and an effective amount of one or more of Compound (B), or a pharmaceutically acceptable salt thereof, wherein: the Compound (C) has the structure:

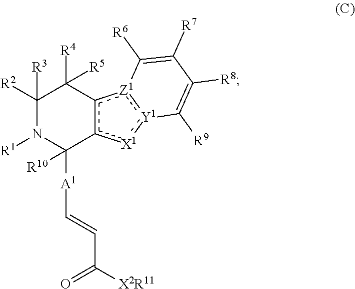

wherein: X¹, Y¹ and Z¹ can be each independently C or N; with the first proviso that at least one of X¹, Y¹ and Z¹ is N; with the second proviso that each of X¹, Y¹ and Z¹ is uncharged; with third proviso that two of the dotted lines indicate double bonds; with the fourth proviso that the valencies of X¹, Y¹ and Z¹ can be each independently satisfied by attachment to a substituent selected from H and R¹²; X² can be O; A¹ can be selected from an optionally substituted cycloalkyl, an optionally substituted aryl, an optionally substituted heteroaryl and an optionally substituted heterocyclyl; R¹ can be selected from an optionally substituted C₁₋₆ alkyl, an optionally substituted cycloalkyl, an optionally substituted cycloalkenyl, an optionally substituted aryl, an optionally substituted heteroaryl, an optionally substituted heterocyclyl, an optionally substituted cycloalkyl(C₁₋₆ alkyl), an optionally substituted cycloalkenyl(C₁₋₆ alkyl), an optionally substituted aryl(C₁₋₆ alkyl), an optionally substituted heteroaryl(C₁₋₆ alkyl) and an optionally substituted heterocyclyl(C₁₋₆ alkyl); R² and R³ can be each independently selected from hydrogen, halogen, an optionally substituted C₁₋₆ alkyl and an optionally substituted C₁₋₆ haloalkyl; or R² and R³ together with the carbon to which R² and R³ are attached can form an optionally substituted cycloalkyl, an optionally substituted cycloalkenyl or an optionally substituted heterocyclyl; R⁴ and R⁵ can be each independently selected from hydrogen, halogen, an optionally substituted C₁₋₆ alkyl and an optionally substituted C₁₋₆ haloalkyl; or R⁴ and R⁵ together with the carbon to which R⁴ and R⁵ are attached can form an optionally substituted cycloalkyl, an optionally substituted cycloalkenyl or an optionally substituted heterocyclyl; R⁶, R⁷, R⁸ and R⁹ can be each independently selected from hydrogen, halogen, hydroxy, an optionally substituted alkyl, an optionally substituted alkoxy, an optionally substituted haloalkyl, an optionally substituted mono-substituted amine, and an optionally substituted di-substituted amine; R¹⁰ can be hydrogen, halogen, an optionally substituted alkyl, or an optionally substituted cycloalkyl; R¹¹ can be hydrogen; and R¹² can be hydrogen, halogen, an optionally substituted C₁₋₃ alkyl, an optionally substituted C₁₋₃ haloalkyl or an optionally substituted C₁₋₃ alkoxy; provided

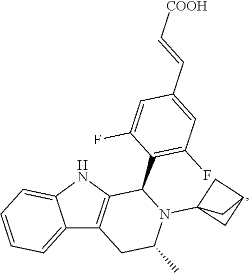 [SMILES: C[C@@H]1Cc2c([nH]c3ccccc23)[C@@H](c2c(F)cc(/C=C/C(=O)O)cc2F)N1C12CC(C1)C2]

that the Compound (C) cannot be or a pharmaceutically acceptable salt thereof; and the one or more of Compound (B) can be a WEE1 inhibitor, or a pharmaceutically acceptable salt thereof.

In some embodiments, for Compound (C), or a pharmaceutically acceptable salt thereof, when X¹ is NH; Y¹ and Z¹ are each C; A¹ is a phenyl, 2-fluorophenyl or 2,6-difluorophenyl; R² and R³ are each methyl or one of R² and R³ is hydrogen and the other of R² and R³ is methyl; and R⁴, R⁵, R⁶, R⁷, R⁸, R⁹ and R¹⁰ are each hydrogen; then R¹ cannot be 2-hydroxyethyl, 2-methylpropyl, 2-fluoro-2-methylpropyl, 3-fluoro-2-methylpropyl, 3-hydroxy-2-methylpropyl or 2-fluoro-3-hydroxy-2-methylpropyl. In other embodiments, for Compound (C), or a pharmaceutically acceptable salt thereof, when R¹⁰ is hydrogen, X¹ is NH, Y¹ and Z¹ are each C, A¹ is an optionally substituted phenyl, one of R² and R³ is hydrogen or an optionally substituted C₁₋₆ alkyl and the other of R² and R³ is an optionally substituted C₁₋₆ alkyl, then R¹ cannot be a substituted C₁₋₆ alkyl substituted with one or more substituents selected from the group consisting of halogen and hydroxy.

In some embodiments, A¹ can be an optionally substituted aryl. For example, A¹ can be an optionally substituted phenyl. Thus, A¹ can be a substituted phenyl or an unsubstituted phenyl. In other embodiments, A¹ can be an optionally substituted cycloalkyl, such as an optionally substituted bicyclopentyl.

In some embodiments, R¹ can be selected from an optionally substituted C₁₋₆ alkyl, an optionally substituted cycloalkyl, an optionally substituted cycloalkyl(C₁₋₆ alkyl), an optionally substituted heterocyclyl and an optionally substituted heterocyclyl(C₁₋₆ alkyl).

In some embodiments, R¹ can be a substituted cycloalkyl. In some embodiments, R¹ is substituted cycloalkyl that can be substituted with one or more substituents selected from halogen, hydroxy, haloalkyl, an optionally substituted alkyl, an optionally substituted cycloalkyl, a substituted alkoxy, a substituted mono-substituted amine and a substituted di-substituted amine. In some embodiments, R¹ can be an optionally substituted cycloalkyl selected from unsubstituted cyclobutyl, unsubstituted difluorocyclobutyl, unsubstituted cyclopentyl and unsubstituted bicyclopentyl. In other embodiments, R¹ can be an optionally substituted cycloalkyl(C₁₋₆ alkyl) selected from unsubstituted cyclopropylmethyl, unsubstituted bicyclopentylmethyl, unsubstituted fluorocyclopropylmethyl, unsubstituted fluorocyclobutylmethyl, unsubstituted methoxycyclopropylmethyl and unsubstituted trifluoromethylcyclopropylmethyl. In still other embodiments, R¹ can be an optionally substituted heterocyclyl selected from unsubstituted tetrahydropyranyl, unsubstituted tetrahydrofuranyl, and unsubstituted oxetanyl. In yet still other embodiments, R¹ is an optionally substituted heterocyclyl(C₁₋₆ alkyl) can be selected from unsubstituted oxetanylmethyl and unsubstituted fluorooxetanylmethyl

In some embodiments, R¹ can be a substituted alkyl. In some embodiments, R¹ can be a substituted alkyl that is substituted with one or more substituents selected from halogen, hydroxy, haloalkyl, an optionally substituted cycloalkyl, a substituted alkoxy, a substituted mono-substituted amine and a substituted di-substituted amine. For example, R¹ can be a substituted alkyl that is a haloalkyl. In some embodiments, R¹ can be an optionally substituted C₁₋₆ alkyl selected from C₄ alkyl, fluoro(C₄ alkyl), and trifluoro(C₂ alkyl).

In some embodiments, R² and R³ can be each independently selected from hydrogen, halogen, an optionally substituted C₁₋₆ alkyl and an optionally substituted C₁₋₆ haloalkyl. In other embodiments, R² and R³ together with the carbon to which R² and R³ are attached can form an optionally substituted cycloalkyl, an optionally substituted cycloalkenyl or an optionally substituted heterocyclyl. In some embodiments, R² can be selected from hydrogen, methyl, fluoromethyl and difluoromethyl.

In some embodiments R⁴ and R⁵ can be each independently selected from hydrogen, halogen, an optionally substituted C₁₋₆ alkyl and an optionally substituted C₁₋₆ haloalkyl. In other embodiments, R⁴ and R⁵ together with the carbon to which R⁴ and R⁵ are attached can form an optionally substituted cycloalkyl, an optionally substituted cycloalkenyl or an optionally substituted heterocyclyl.

In some embodiments, R⁷ can be selected from halogen, hydroxy and unsubstituted alkoxy. For example, in some embodiments, R⁷ can be selected from fluoro and methoxy.

In some embodiments, R¹² can be hydrogen. In other embodiments, R¹² can be not hydrogen.

In some embodiments, Compound (A), or a pharmaceutically acceptable salt thereof (including one or more pharmaceutically acceptable salt forms, such as those described herein), can be used in combination with one or more WEE1 inhibitors, or a pharmaceutically acceptable salt thereof. In some embodiments, Compound (C), or a pharmaceutically acceptable salt thereof, can be used in combination with one or more WEE1 inhibitors, or a pharmaceutically acceptable salt thereof.

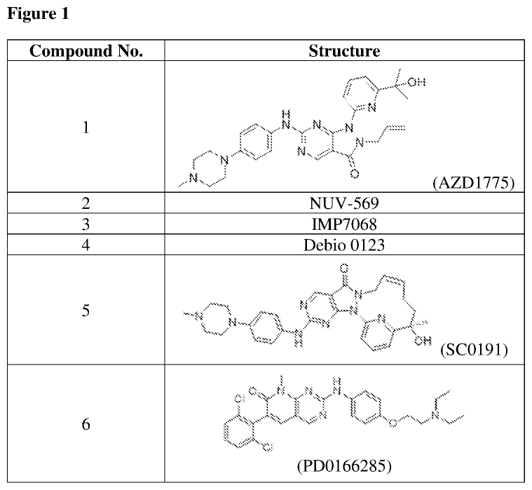

A non-limiting list of WEE1 inhibitors are described herein, and include those provided in FIG. 1. Additional WEE1 inhibitors are provided in WO 2007/126122, WO 2008/133866, WO 2011/034743, WO 2019/138227, WO 2018/162932, WO 2018/011570, WO 2018/011569, WO 2015/092431, WO 2015/019037, WO 2014/167347. WO 2020/210375. WO 2020/210377, WO 2020/210380 WO 2020/210381, WO 2020/210383, WO 2019/011228, WO 2018/090939, WO 2020/221358, WO 2019/085933, EP 3712150, WO 2019/085933 and WO 96/34867, each of which is hereby incorporated by reference for the limited purpose of their disclosure of compounds that are WEE1 inhibitors. In some embodiments, the WEE1 inhibitor can be AZD 1775. In some embodiments, the WEE1 inhibitor can be NUV-569. In some embodiments, the WEE1 inhibitor can be IMP7068. In some embodiments, the WEE1 inhibitor can be Debio 0123. In some embodiments, the WEE1 inhibitor can be SC0191. In some embodiments, the WEE1 inhibitor can be PD-166285.

Examples of Compound (C) include the following:

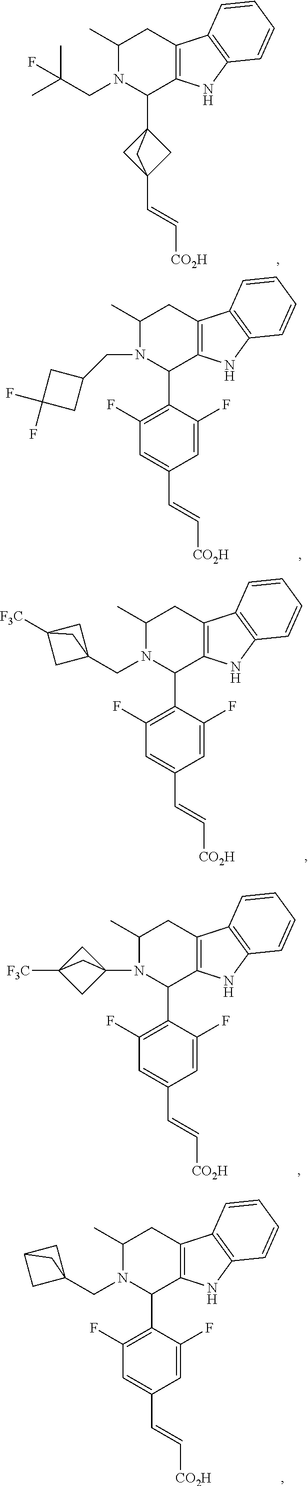

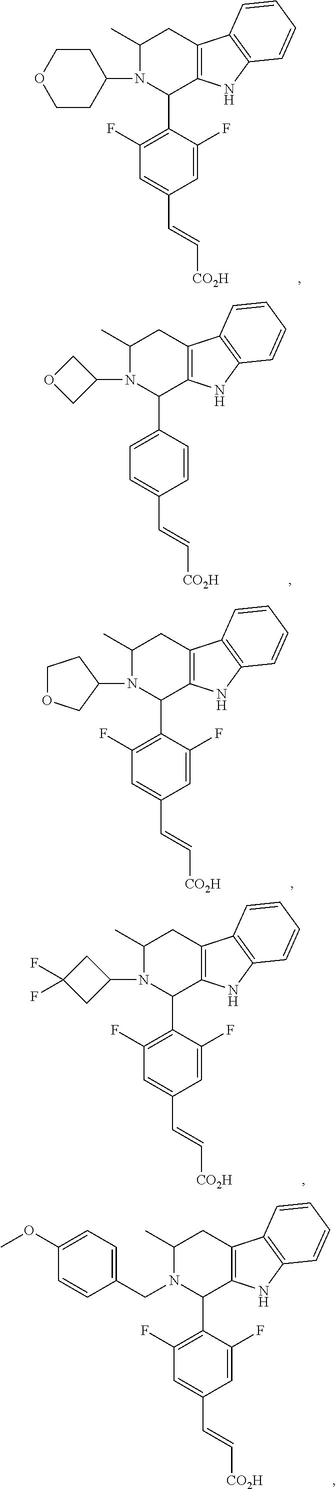

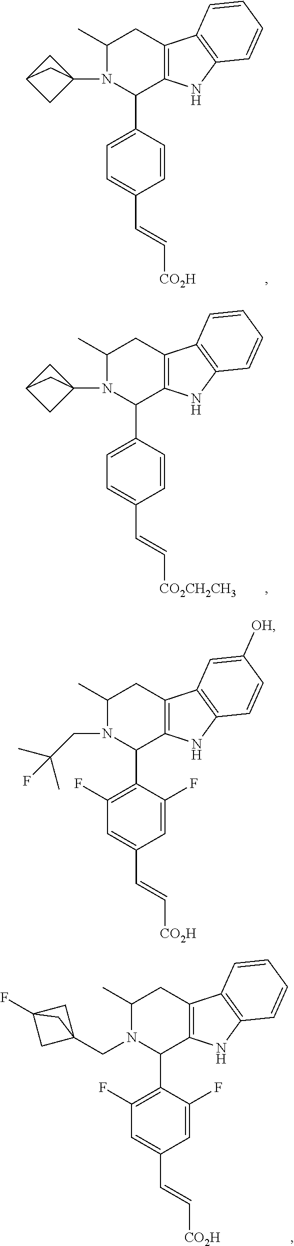

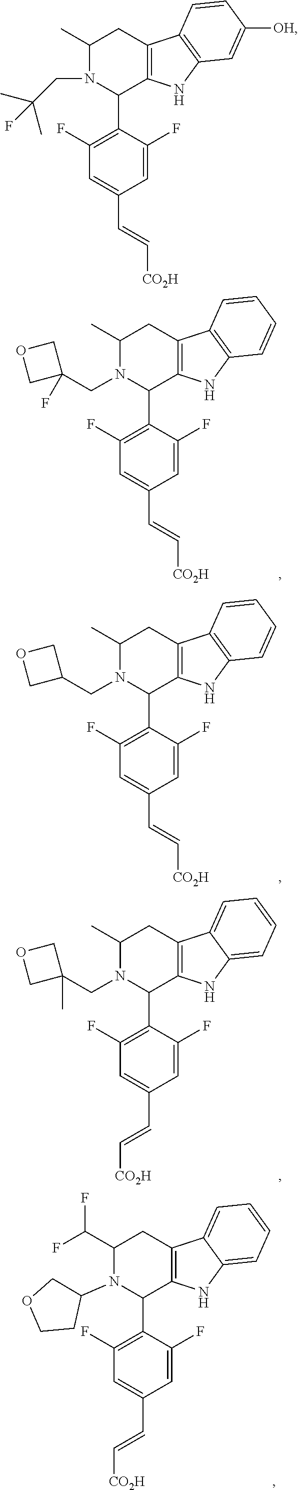

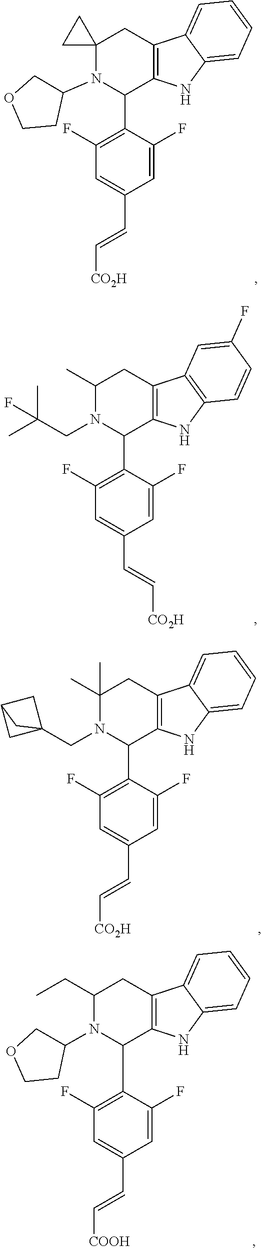 [SMILES: O=C(O)/C=C/c1cc(F)c(C2c3[nH]c4ccccc4c3CC3(CC3)N2C2CCOC2)c(F)c1 ; CC1Cc2c([nH]c3ccc(F)cc23)C(c2c(F)cc(/C=C/C(=O)O)cc2F)N1CC(C)(C)F ; CC1(C)Cc2c([nH]c3ccccc23)C(c2c(F)cc(/C=C/C(=O)O)cc2F)N1CC12CC(C1)C2 ; CCC1Cc2c([nH]c3ccccc23)C(c2c(F)cc(/C=C/C(=O)O)cc2F)N1C1CCOC1]

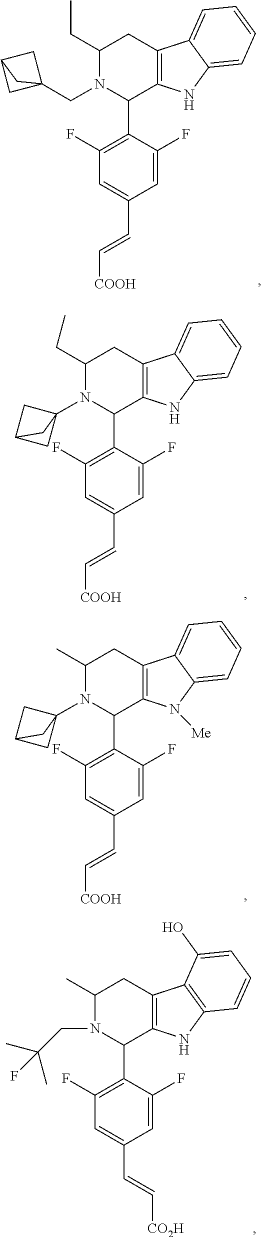

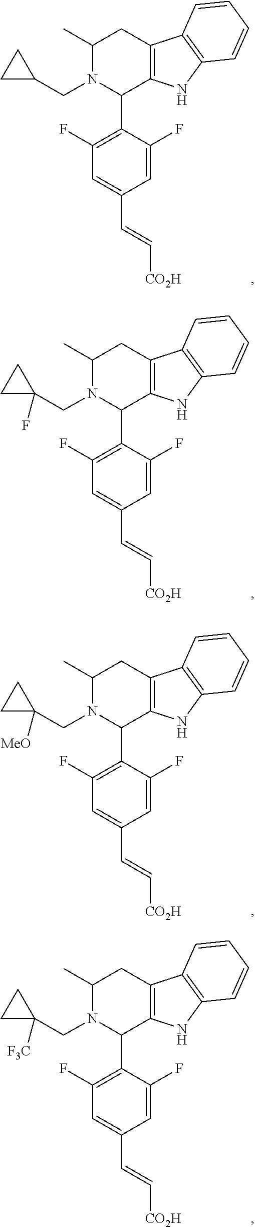 [SMILES: CC1Cc2c([nH]c3ccccc23)C(c2c(F)cc(/C=C/C(=O)O)cc2F)N1CC1CC1 ; CC1Cc2c([nH]c3ccccc23)C(c2c(F)cc(/C=C/C(=O)O)cc2F)N1CC1(F)CC1 ; COC1(CN2C(C)Cc3c([nH]c4ccccc34)C2c2c(F)cc(/C=C/C(=O)O)cc2F)CC1 ; CC1Cc2c([nH]c3ccccc23)C(c2c(F)cc(/C=C/C(=O)O)cc2F)N1CC1(C(F)(F)F)CC1]

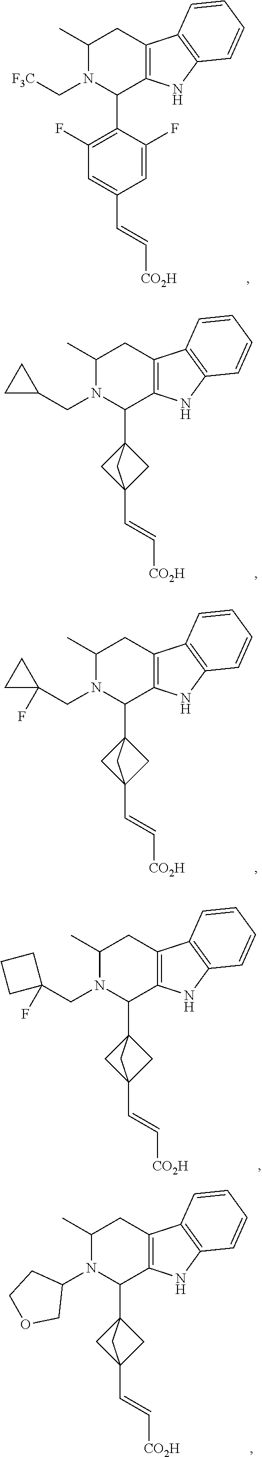

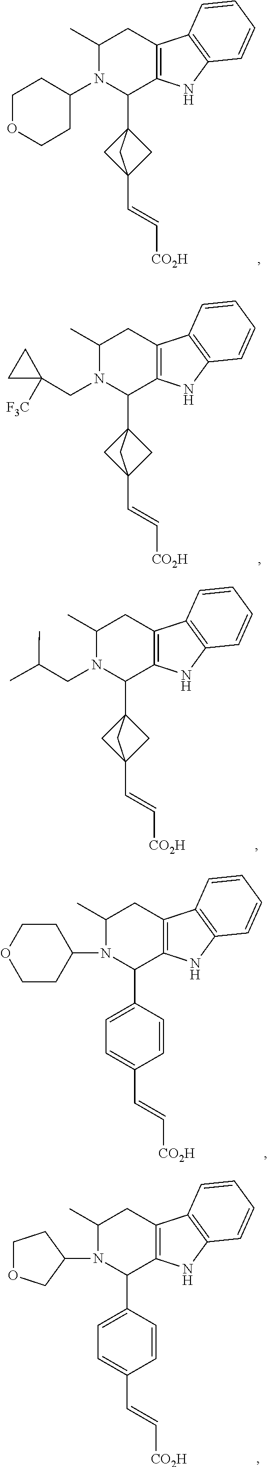 [SMILES: CC1Cc2c([nH]c3ccccc23)C(C23CC(/C=C/C(=O)O)(C2)C3)N1C1CCOCC1 ; CC1Cc2c([nH]c3ccccc23)C(C23CC(/C=C/C(=O)O)(C2)C3)N1CC1(C(F)(F)F)CC1 ; CC(C)CN1C(C)Cc2c([nH]c3ccccc23)C1C12CC(/C=C/C(=O)O)(C1)C2 ; CC1Cc2c([nH]c3ccccc23)C(c2ccc(/C=C/C(=O)O)cc2)N1C1CCOCC1 ; CC1Cc2c([nH]c3ccccc23)C(c2ccc(/C=C/C(=O)O)cc2)N1C1CCOC1]

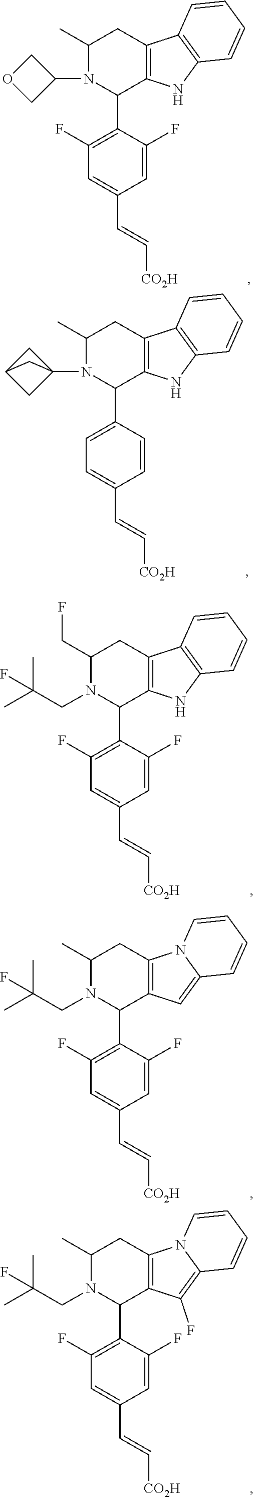 [SMILES: CC1Cc2c([nH]c3ccccc23)C(c2c(F)cc(/C=C/C(=O)O)cc2F)N1C1COC1 ; CC1Cc2c([nH]c3ccccc23)C(c2ccc(/C=C/C(=O)O)cc2)N1C12CC(C1)C2 ; CC(C)(F)CN1C(CF)Cc2c([nH]c3ccccc23)C1c1c(F)cc(/C=C/C(=O)O)cc1F ; CC1Cc2c(cc3ccccn23)C(c2c(F)cc(/C=C/C(=O)O)cc2F)N1CC(C)(C)F ; CC1Cc2c(c(F)c3ccccn23)C(c2c(F)cc(/C=C/C(=O)O)cc2F)N1CC(C)(C)F]

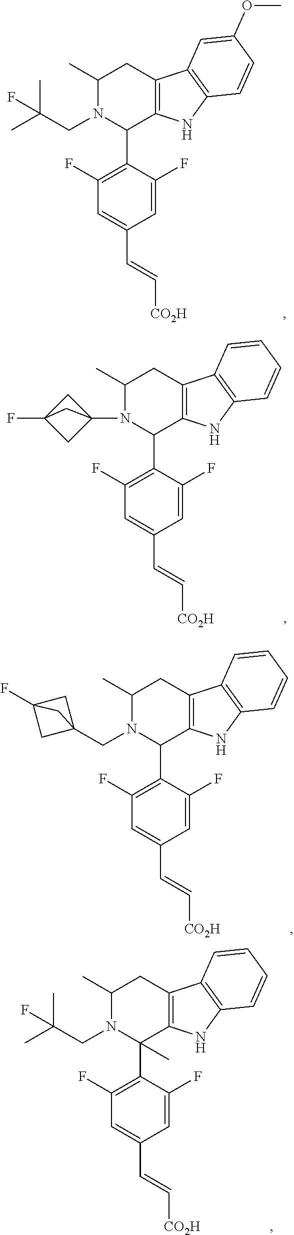

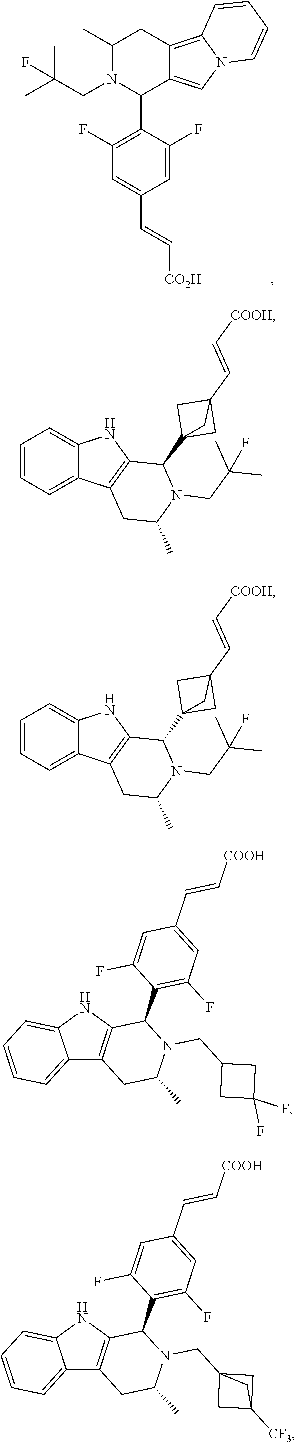 [SMILES: CC1Cc2c(cn3ccccc23)C(c2c(F)cc(/C=C/C(=O)O)cc2F)N1CC(C)(C)F ; C[C@@H]1Cc2c([nH]c3ccccc23)[C@@H](C23CC(/C=C/C(=O)O)(C2)C3)N1CC(C)(C)F ; C[C@@H]1Cc2c([nH]c3ccccc23)[C@H](C23CC(/C=C/C(=O)O)(C2)C3)N1CC(C)(C)F ; C[C@@H]1Cc2c([nH]c3ccccc23)[C@@H](c2c(F)cc(/C=C/C(=O)O)cc2F)N1CC1CC(F)(F)C1 ; C[C@@H]1Cc2c([nH]c3ccccc23)[C@@H](c2c(F)cc(/C=C/C(=O)O)cc2F)N1CC12CC(C(F)(F)F)(C1)C2]

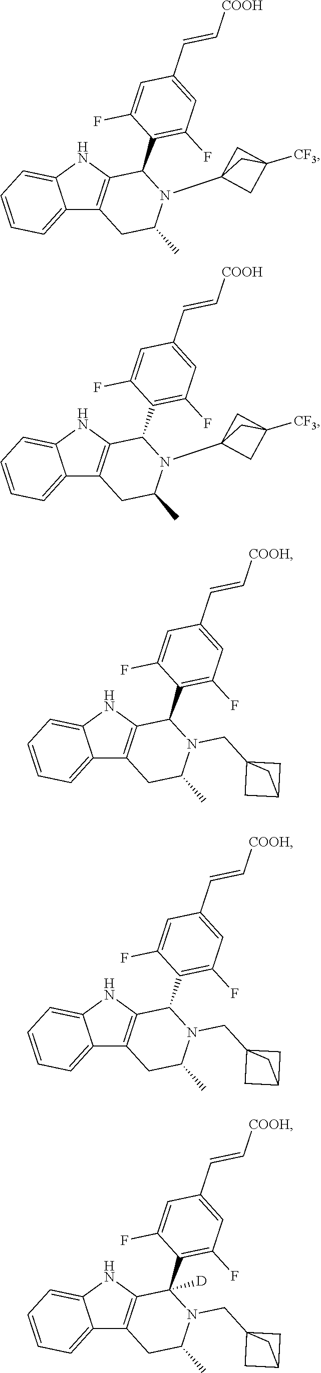

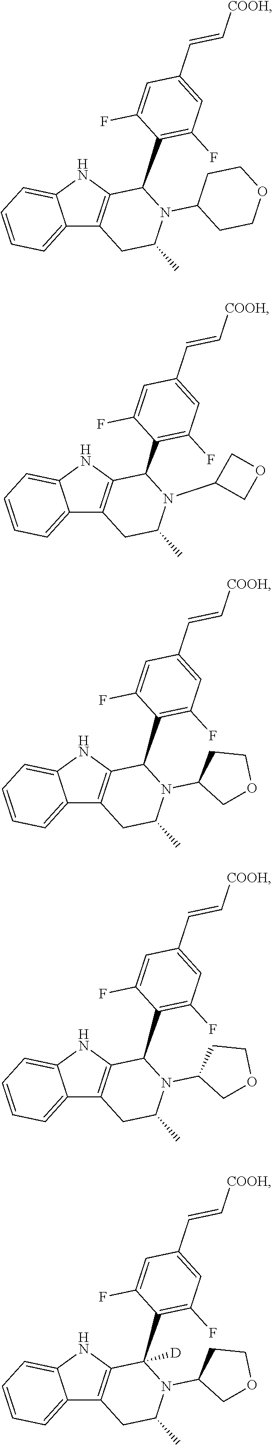 [SMILES: C[C@@H]1Cc2c([nH]c3ccccc23)[C@@H](c2c(F)cc(/C=C/C(=O)O)cc2F)N1C1CCOCC1 ; C[C@@H]1Cc2c([nH]c3ccccc23)[C@@H](c2c(F)cc(/C=C/C(=O)O)cc2F)N1C1COC1 ; C[C@@H]1Cc2c([nH]c3ccccc23)[C@@H](c2c(F)cc(/C=C/C(=O)O)cc2F)N1[C@H]1CCOC1 ; C[C@@H]1Cc2c([nH]c3ccccc23)[C@@H](c2c(F)cc(/C=C/C(=O)O)cc2F)N1[C@@H]1CCOC1 ; [2H][C@@]1(c2c(F)cc(/C=C/C(=O)O)cc2F)c2[nH]c3ccccc3c2C[C@@H](C)N1[C@H]1CCOC1]

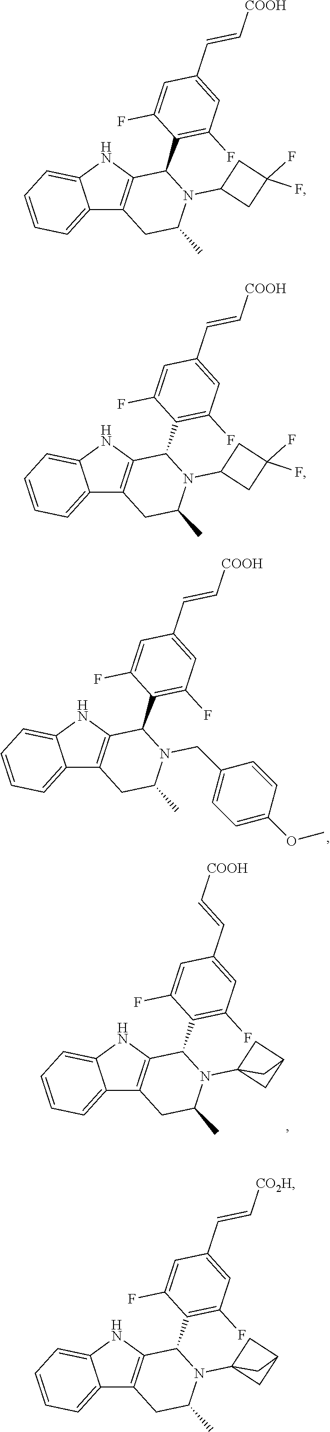

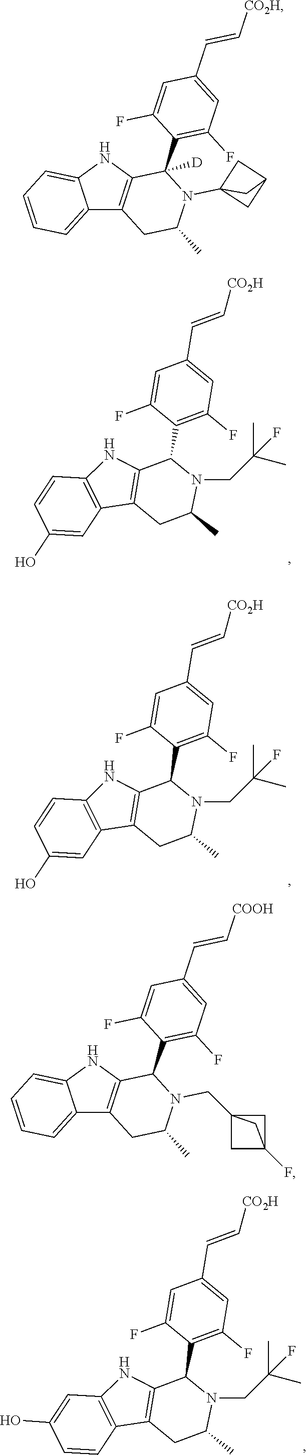

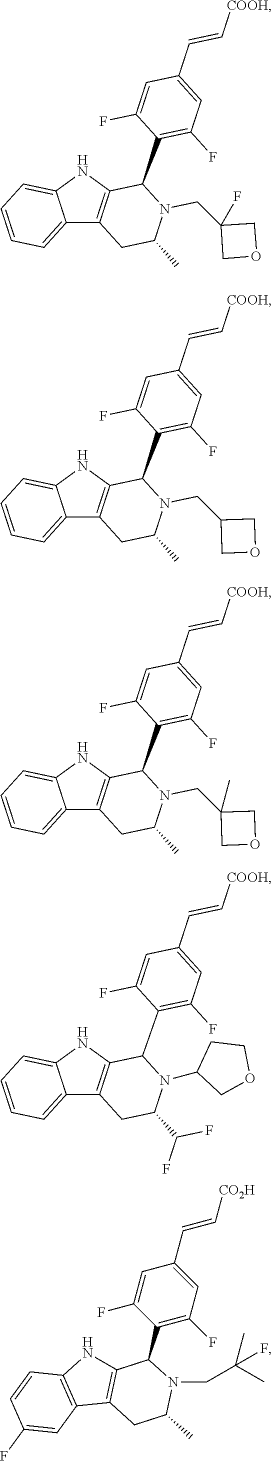

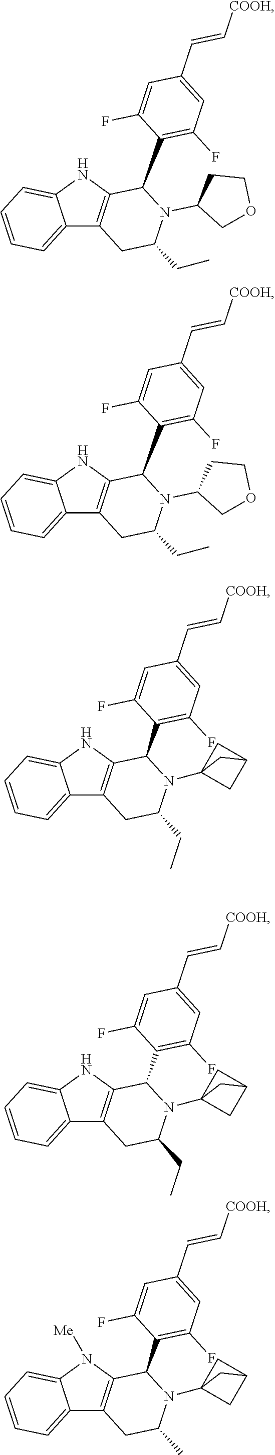

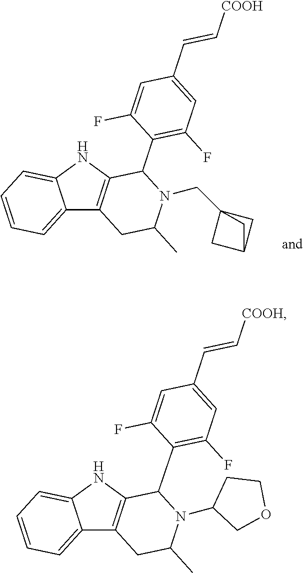 [SMILES: CC1Cc2c([nH]c3ccccc23)C(c2c(F)cc(/C=C/C(=O)O)cc2F)N1CC12CC(C1)C2 ; CC1Cc2c([nH]c3ccccc23)C(c2c(F)cc(/C=C/C(=O)O)cc2F)N1C1CCOC1]

or a pharmaceutically acceptable salt of any of the foregoing.

Compound (A), along with pharmaceutically acceptable salts thereof, can be prepared as described herein and in WO 2017/172957, which is hereby incorporated by reference in its entirety. As described in WO 2017/172957, Compound (A) is an estrogen receptor alpha (ERα) inhibitor.

Embodiments of combinations of Compound (A), including pharmaceutically acceptable salts and salt forms thereof (such as Form A and/or Form C), and Compound (B), including pharmaceutically acceptable salts thereof, are provided in Table 1. In Table 1, “A” represents Compound (A), including pharmaceutically acceptable salts and salt forms thereof, and the numbers represent a compound as provided in FIG. 1, including pharmaceutically acceptable salts thereof.

TABLE 1
Cmpd:Cmpd
1:A
2:A
3:A
4:A
5:A
6:A

The order of administration of compounds in a combination described herein can vary. In some embodiments, Compound (A), including pharmaceutically acceptable salts and salt forms thereof, and/or Compound (C), including pharmaceutically acceptable salts thereof, can be administered prior to all of Compound (B), or a pharmaceutically acceptable salt thereof. In other embodiments, Compound (A), including pharmaceutically acceptable salts and salt forms thereof, and/or Compound (C), including pharmaceutically acceptable salts thereof, can be administered prior to at least one Compound (B), or a pharmaceutically acceptable salt thereof. In still other embodiments, Compound (A), including pharmaceutically acceptable salts and salt forms thereof, and/or Compound (C), including pharmaceutically acceptable salts thereof, can be administered concomitantly with Compound (B), or a pharmaceutically acceptable salt thereof. In yet still other embodiments, Compound (A), including pharmaceutically acceptable salts and salt forms thereof, and/or Compound (C), including pharmaceutically acceptable salts thereof, can be administered subsequent to the administration of at least one Compound (B), or a pharmaceutically acceptable salt thereof. In some embodiments, Compound (A), including pharmaceutically acceptable salts and salt forms thereof, and/or Compound (C), including pharmaceutically acceptable salts thereof, can be administered subsequent to the administration of all Compound (B), or a pharmaceutically acceptable salt thereof.

There may be several advantages for using a combination of compounds described herein. For example, combining compounds that attack multiple pathways at the same time, can be more effective in treating a cancer, such as those described herein, compared to when the compounds of combination are used as monotherapy.

In some embodiments, a combination as described herein of Compound (A), including pharmaceutically acceptable salts and salt forms thereof, and one or more of Compound (B), or pharmaceutically acceptable salts thereof, can decrease the number and/or severity of side effects that can be attributed to a compound described herein, such as Compound (B), or a pharmaceutically acceptable salt thereof. In other embodiments, a combination as described herein of Compound (C), including pharmaceutically acceptable salts thereof, and one or more of Compound (B), or pharmaceutically acceptable salts thereof, can decrease the number and/or severity of side effects that can be attributed to Compound (B), or a pharmaceutically acceptable salt thereof.

Using a combination of compounds described herein can results in additive, synergistic or strongly synergistic effect. A combination of compounds described herein can result in an effect that is not antagonistic.

In some embodiments, a combination as described herein of Compound (A), including pharmaceutically acceptable salts and salt forms thereof, and one or more of Compound (B), or pharmaceutically acceptable salts thereof, can result in an additive effect. In other embodiments, a combination as described herein of Compound (C), including pharmaceutically acceptable salts thereof, and one or more of Compound (B), or pharmaceutically acceptable salts thereof, can result in an additive effect.

In some embodiments, a combination as described herein of Compound (A), including pharmaceutically acceptable salts and salt forms thereof, and one or more of Compound (B), or pharmaceutically acceptable salts thereof, can result in a synergistic effect. In other embodiments, a combination as described herein of Compound (C), including pharmaceutically acceptable salts thereof, and one or more of Compound (B), or pharmaceutically acceptable salts thereof, can result in a synergistic effect.

In some embodiments, a combination as described herein of Compound (A), including pharmaceutically acceptable salts and salt forms thereof, and one or more of Compound (B), or pharmaceutically acceptable salts thereof, can result in a strongly synergistic effect. In other embodiments, a combination as described herein of Compound (C), including pharmaceutically acceptable salts thereof, and one or more of Compound (B), or pharmaceutically acceptable salts thereof, can result in a strongly synergistic effect.

In some embodiments, a combination as described herein of Compound (A), including pharmaceutically acceptable salts and salt forms thereof, and one or more of Compound (B), or pharmaceutically acceptable salts thereof, is not antagonistic. In other embodiments, a combination as described herein of Compound (C), including pharmaceutically acceptable salts thereof, and one or more of Compound (B), or pharmaceutically acceptable salts thereof, is not antagonistic.

As used herein, the term “antagonistic” means that the activity of the combination of compounds is less compared to the sum of the activities of the compounds in combination when the activity of each compound is determined individually (i.e., as a single compound). As used herein, the term “synergistic effect” means that the activity of the combination of compounds is greater than the sum of the individual activities of the compounds in the combination when the activity of each compound is determined individually. As used herein, the term “additive effect” means that the activity of the combination of compounds is about equal to the sum of the individual activities of the compounds in the combination when the activity of each compound is determined individually.

A potential advantage of utilizing a combination as described herein may be a reduction in the required amount(s) of the compound(s) that is effective in treating a disease condition disclosed herein compared to when each compound is administered as a monotherapy. For example, the amount of Compound (B), or a pharmaceutically acceptable salt thereof, used in a combination described herein can be less compared to the amount of Compound (B), or a pharmaceutically acceptable salt thereof, needed to achieve the same reduction in a disease marker (for example, tumor size) when administered as a monotherapy. Another potential advantage of utilizing a combination as described herein is that the use of two or more compounds having different mechanisms of action can create a higher barrier to the development of resistance compared to when a compound is administered as monotherapy. Additional advantages of utilizing a combination as described herein may include little to no cross resistance between the compounds of a combination described herein; different routes for elimination of the compounds of a combination described herein; and/or little to no overlapping toxicities between the compounds of a combination described herein.

### Pharmaceutical Compositions

Compound (A), including pharmaceutically acceptable salts and salt forms thereof, can be provided in a pharmaceutical composition. Compound (B), including pharmaceutically acceptable salts thereof, can be provided in a pharmaceutical composition. Similarly, Compound (C), including pharmaceutically acceptable salts thereof, can be provided in a pharmaceutical composition.

The term “pharmaceutical composition” refers to a mixture of one or more compounds and/or salts disclosed herein with other chemical components, such as diluents, carriers and/or excipients. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, and salicylic acid. Pharmaceutical compositions will generally be tailored to the specific intended route of administration.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. For example, stabilizers such as anti-oxidants and metal-chelating agents are excipients. In an embodiment, the pharmaceutical composition comprises an anti-oxidant and/or a metal-chelating agent. A “diluent” is a type of excipient.

In some embodiments, Compounds (B), along with pharmaceutically acceptable salts thereof, can be provided in a pharmaceutical composition that includes Compound (A), including pharmaceutically acceptable salts and salt forms thereof, and/or Compound (C), including pharmaceutically acceptable salts thereof. In other embodiments, Compound (B), along with pharmaceutically acceptable salts thereof, can be administered in a pharmaceutical composition that is separate from a pharmaceutical composition that includes Compound (A), including pharmaceutically acceptable salts and salt forms thereof. In still other embodiments, Compounds (B), along with pharmaceutically acceptable salts thereof, can be administered in a pharmaceutical composition that is separate from a pharmaceutical composition that includes Compound (C), including pharmaceutically acceptable salts thereof.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, Compound (A), including pharmaceutically acceptable salts and salt forms thereof, can be administered orally. In some embodiments, Compound (C), including pharmaceutically acceptable salts thereof, can be administered orally. In some embodiments, Compound (A), including pharmaceutically acceptable salts and salt forms thereof, can be provided to a subject by the same route of administration as Compound (B), along with pharmaceutically acceptable salts thereof. In other embodiments, Compound (A), including pharmaceutically acceptable salts and salt forms thereof, can be provided to a subject by a different route of administration as Compound (B), along with pharmaceutically acceptable salts thereof. In still other embodiments, Compound (C), including pharmaceutically acceptable salts thereof, can be provided to a subject by the same route of administration as Compound (B), along with pharmaceutically acceptable salts thereof. In yet still other embodiments, Compound (C), including pharmaceutically acceptable salts thereof, can be provided to a subject by a different route of administration as Compound (B), along with pharmaceutically acceptable salts thereof.

One may also administer the compound, salt and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical carrier may also be prepared, placed in an appropriate container, and labeled for treatment of an indicated condition.

### Uses and Methods of Treatment

As provided herein, in some embodiments, a combination of compounds that includes an effective amount of Compound (A), including pharmaceutically acceptable salts and salt forms thereof, and an effective amount of one or more of Compound (B), or a pharmaceutically acceptable salt thereof, can be used to treat a disease or condition. In some embodiments, a combination of compounds that includes an effective amount of Compound (C), including pharmaceutically acceptable salts thereof, and an effective amount of one or more of Compound (B), or a pharmaceutically acceptable salt thereof, can be used to treat a disease or condition.

In some embodiments, the disease or condition can be selected from a breast cancer, a cervical cancer, an ovarian cancer, an uterine cancer, a vaginal cancer, a vulvar cancer, a brain cancer, a cervicocerebral cancer, an esophageal cancer, a thyroid cancer, a small cell cancer, a non-small cell cancer, a lung cancer, a stomach cancer, a gallbladder/bile duct cancer, a liver cancer, a pancreatic cancer, a colon cancer, a rectal cancer, a choriocarcinoma, an uterus body cancer, an uterocervical cancer, a renal pelvis/ureter cancer, a bladder cancer, a prostate cancer, a penis cancer, a testicular cancer, a fetal cancer, a Wilms' cancer, a skin cancer, a malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma. In other embodiments, the disease or condition can be selected from a breast cancer, a cervical cancer, an ovarian cancer, an uterine cancer, a vaginal cancer, and a vulvar cancer.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject can be human. In some embodiments, the subject can be a child and/or an infant, for example, a child or infant with a fever. In other embodiments, the subject can be an adult.

As used herein, the terms “treat,” “treating,” “treatment,” “therapeutic,” and “therapy” do not necessarily mean total cure or abolition of the disease or condition. Any alleviation of any undesired signs or symptoms of the disease or condition, to any extent can be considered treatment and/or therapy. Furthermore, treatment may include acts that may worsen the subject's overall feeling of well-being or appearance.

The term “effective amount” is used to indicate an amount of an active compound, or pharmaceutical agent, that elicits the biological or medicinal response indicated. For example, an effective amount of compound, salt or composition can be the amount needed to prevent, alleviate or ameliorate symptoms of the disease or condition, or prolong the survival of the subject being treated. This response may occur in a tissue, system, animal or human and includes alleviation of the signs or symptoms of the disease or condition being treated. Determination of an effective amount is well within the capability of those skilled in the art, in view of the disclosure provided herein. The effective amount of the compounds disclosed herein required as a dose will depend on the route of administration, the type of animal, including human, being treated and the physical characteristics of the specific animal under consideration. The dose can be tailored to achieve a desired effect, but will depend on such factors as weight, diet, concurrent medication and other factors which those skilled in the medical arts will recognize.

For example, an effective amount of a compound, or radiation, is the amount that results in: (a) the reduction, alleviation or disappearance of one or more symptoms caused by the cancer, (b) the reduction of tumor size, (c) the elimination of the tumor, and/or (d) long-term disease stabilization (growth arrest) of the tumor.

Various types of breast cancer are known. In some embodiments, the breast cancer can be ER positive breast cancer. In some embodiments, the breast cancer can be ER positive, HER2-negative breast cancer. In some embodiments, the breast cancer can be local breast cancer (as used herein, “local” breast cancer means the cancer has not spread to other areas of the body). In other embodiments, the breast cancer can be metastatic breast cancer. A subject can have a breast cancer that has not been previously treated.

In some cases, following breast cancer treatment, a subject can relapse or have reoccurrence of breast cancer. As used herein, the terms “relapse” and “reoccurrence” are used in their normal sense as understood by those skilled in the art. Thus, the breast cancer can be recurrent breast cancer. In some embodiments, the subject has relapsed after a previous treatment for breast cancer. For example, the subject has relapsed after receiving one or more treatments with a SERM, a SERD and/or aromatase inhibitor, such as those described herein.

Within ESR1, several amino acid mutations have been identified. Mutations in ESR1 have been proposed as playing a role in resistance. There are several therapies for inhibiting estrogen receptors, including selective ER modulators (SERM), selective ER degraders (SERD) and aromatase inhibitors. One issue that can arise from the aforementioned cancer therapies is the development of resistance to the cancer therapy. Acquired resistance to cancer therapy, such as endocrine therapy, has been noted in nearly one-third of women treated with tamoxifen and other endocrine therapies. See Alluri et al., “Estrogen receptor mutations and their role in breast cancer progression” Breast Cancer Research (2014) 16:494. Researchers have suspected mutations in the estrogen receptor as one of the reasons for acquired resistance to cancer therapy, such as endocrine therapy. Thus, there is a need for compounds that can treat breast cancer wherein the cancer has one or more mutations within ESR1.

Some embodiments disclosed herein are relate to the use of a combination of compounds that includes an effective amount of Compound (A), including pharmaceutically acceptable salts and salt forms thereof, and an effective amount of one or more of Compound (B), or a pharmaceutically acceptable salt thereof, in the manufacture for a medicament for treating breast cancer in a subject in need thereof, wherein the breast cancer has at least one point mutation within the Estrogen Receptor 1 (ESR1) that encodes Estrogen receptor alpha (ERα). Other embodiments relate herein are directed to the use of a combination of compounds that includes an effective amount of Compound (A), including pharmaceutically acceptable salts and salt forms thereof, and an effective amount of one or more of Compound (B), or a pharmaceutically acceptable salt thereof, for treating breast cancer in a subject in need thereof, wherein the breast cancer has at least one point mutation within the Estrogen Receptor 1 (ESR1) that encodes Estrogen receptor alpha (ERα). Still other embodiments disclosed herein are relate to a method of treating breast cancer in a subject in need thereof with a combination of compounds that includes an effective amount of Compound (A), including pharmaceutically acceptable salts and salt forms thereof, and an effective amount of one or more of Compound (B), or a pharmaceutically acceptable salt thereof, wherein the breast cancer has at least one point mutation within the Estrogen Receptor 1 (ESR1) that encodes Estrogen receptor alpha (ERα).

In some embodiments, the mutation can be in the ligand binding domain (LBD) of ESR1. In some embodiments, one or more mutations can be at an amino acid selected from: A593, S576, G557, R555, L549, A546, E542, L540, D538, Y537, L536, P535, V534, V533, N532, K531, C530, H524, E523, M522, R503, L497, K481, V478, R477, E471, S463, F461, S432, G420, V418, D411, L466, S463, L453, G442, M437, M421, M396, V392, M388, E380, G344, S338, L370, S329, K303, A283, S282, E279, G274, K252, R233, P222, G160, N156, P147, G145, F97, N69, A65, A58 and S47. In some embodiments, one or more mutations can be at an amino acid selected from: D538, Y537, L536, P535, V534, S463, V392 and E380. In some embodiments, one or more mutations can be at an amino acid selected from: D538 and Y537.

In some embodiments, one or more mutations can be selected from: K303R, D538G, Y537S, E380Q, Y537C, Y537N, A283V, A546D, A546T, A58T, A593D, A65V, C530L, D411H, E279V, E471D, E471V, E523Q, E542G, F461V, F97L, G145D, G160D, G274R, G344D, G420D, G442R, G557R, H524L, K252N, K481N, K531E, L370F, L453F, L466Q, L497R, L536H, L536P, L536Q, L536R, L540Q, L549P, M388L, M396V, M421V, M437I, M522I, N156T, N532K, N69K, P147Q, P222S, P535H, R233G, R477Q, R503W, R555H, S282C, S329Y, S338G, S432L, S463P, S47T, S576L, V392I, V418E, V478L, V533M, V534E, Y537D and Y537H.

Some embodiments disclosed herein are relate to the use of a combination of compounds that includes an effective amount of Compound (A), including pharmaceutically acceptable salts and salt forms thereof, and an effective amount of one or more of Compound (B), or a pharmaceutically acceptable salt thereof, in the manufacture for a medicament for treating breast cancer in a subject in need thereof, wherein the breast cancer does not include at least one point mutation (for example, a point mutation within the Estrogen Receptor 1 (ESR1) that encodes Estrogen receptor alpha (ERα)). Other embodiments relate herein are directed to the use of a combination of compounds that includes an effective amount of Compound (A), including pharmaceutically acceptable salts and salt forms thereof, and an effective amount of one or more of Compound (B), or a pharmaceutically acceptable salt thereof, for treating breast cancer in a subject in need thereof, wherein the breast cancer does not include has at least one point mutation, such as a point mutation within the Estrogen Receptor 1 (ESR1) that encodes Estrogen receptor alpha (ERα). Still other embodiments disclosed herein are relate to a method of treating breast cancer in a subject in need thereof with a combination of compounds that includes an effective amount of Compound (A), including pharmaceutically acceptable salts and salt forms thereof, and an effective amount of one or more of Compound (B), or a pharmaceutically acceptable salt thereof, wherein the breast cancer does not include has at least one point mutation within the Estrogen Receptor 1 (ESR1) that encodes Estrogen receptor alpha (ERα) (for example, a point mutation within the Estrogen Receptor 1 (ESR1) that encodes Estrogen receptor alpha (ERα)).

As provided herein, several studies have shown that a potential cause of resistance in ER-positive breast cancer is due to acquired mutations in ESR1 due to endocrine therapy. In some embodiments, the subject had been previously treated with one or more selective ER modulators. For example, subject had been treated previously with one or more selected ER modulators selected from tamoxifen, raloxifene, ospemifene, bazedoxifene, toremifene and lasofoxifene, or a pharmaceutically acceptable salt of any of the foregoing. In some embodiments, the subject had been treated previously with one or more selective ER degraders, such as fulvestrant, (E)-3-[3,5-Difluoro-4-[(1R,3R)-2-(2-fluoro-2-methylpropyl)-3-methyl-1,3,4,9-tetrahydropyrido[3,4-b]indol-1-yl]phenyl]prop-2-enoic acid (AZD9496), (R)-6-(2-(ethyl(4-(2-(ethylamino)ethyl)benzyl)amino)-4-methoxyphenyl)-5,6,7,8-tetrahydronaphthalen-2-ol (elacestrant, RAD1901), (E)-3-(4-((E)-2-(2-chloro-4-fluorophenyl)-1-(1H-indazol-5-yl)but-1-en-1-yl)phenyl)acrylic acid (Brilanestrant, ARN-810, GDC-0810), (E)-3-(4-((2-(2-(1,1-difluoroethyl)-4-fluorophenyl)-6-hydroxybenzo[b]thiophen-3-yl)oxy)phenyl)acrylic acid (LSZ102), (E)-N,N-dimethyl-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide (H3B-6545), (E)-3-(4-((2-(4-fluoro-2,6-dimethylbenzoyl)-6-hydroxybenzo[b]thiophen-3-yl)oxy)phenyl)acrylic acid (rintodestrant, G1T48), D-0502, SHR9549, ARV-471, 3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoropropan-1-ol (giredestrant, GDC-9545), (S)-8-(2,4-dichlorophenyl)-9-(4-((1-(3-fluoropropyl)pyrrolidin-3-yl)oxy)phenyl)-6,7-dihydro-5H-benzo [7]annulene-3-carboxylic acid (SAR439859), N-[1-(3-fluoropropyl)azetidin-3-yl]-6-[(6S,8R)-8-methyl-7-(2,2,2-trifluoroethyl)-6,7,8,9-tetrahydro-3H-pyrazolo[4,3-f]isoquinolin-6-yl]pyridin-3-amine (AZD9833), OP-1250 and LY3484356, or a pharmaceutically acceptable salt of any of the foregoing. In some embodiments, the subject had been treated previously with one or more aromatase inhibitors. The aromatase inhibitors can be a steroidal aromatase inhibitor or a non-steroidal aromatase inhibitor. For example, the one or more aromatase inhibitors can be selected from (exemestane (steroidal aromatase inhibitor), testolactone (steroidal aromatase inhibitor); anastazole (non-steroidal aromatase inhibitor) and letrazole (non-steroidal aromatase inhibitor), including pharmaceutically acceptable salts of any of the foregoing.

In some embodiments, the breast cancer can be present in subject, wherein the subject can be a woman. As women approach middle-age, a woman can be in a stage of menopause. In some embodiments, the subject can be a premenopausal woman. In other embodiments, the subject can be a perimenopausal woman. In still other embodiments, the subject can be a menopausal woman. In yet still other embodiments, the subject can be a postmenopausal woman. In other embodiments, the breast cancer can be present in a subject, wherein the subject can be a man. The serum estradiol level of the subject can vary. In some embodiments, the serum estradiol level (E2) of the subject can be in the range of >15 pg/mL to 350 pg/mL. In other embodiments, the serum estradiol level (E2) of the subject can be ≤15 pg/mL. In other embodiments, the serum estradiol level (E2) of the subject can be ≤10 pg/mL.

The amount of compound, salt and/or composition required for use in treatment will vary not only with the particular compound or salt selected but also with the route of administration, the nature and/or symptoms of the disease or condition being treated and the age and condition of the patient and will be ultimately at the discretion of the attendant physician or clinician. In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the dosage ranges described herein in order to effectively and aggressively treat particularly aggressive diseases or conditions.

As will be readily apparent to one skilled in the art, the useful in vivo dosage to be administered and the particular mode of administration will vary depending upon the age, weight, the severity of the affliction, the mammalian species treated, the particular compounds employed and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies. For example, useful dosages of compounds (A), (B) and/or (C), or pharmaceutically acceptable salts of any of the foregoing, can be determined by comparing their in vitro activity, and in vivo activity in animal models. Such comparison can be done by comparison against an established drug, such as cisplatin and/or gemcitabine)

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the disease or condition to be treated and to the route of administration. The severity of the disease or condition may, for example, be evaluated, in part, by standard prognostic evaluation methods. Further, the dose and perhaps dose frequency, will also vary according to the age, body weight and response of the individual patient. A program comparable to that discussed above may be used in veterinary medicine.

Compounds, salts and compositions disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### EXAMPLES

Additional embodiments are disclosed in further detail in the following examples, which are not in any way intended to limit the scope of the claims.

### CTG Assay

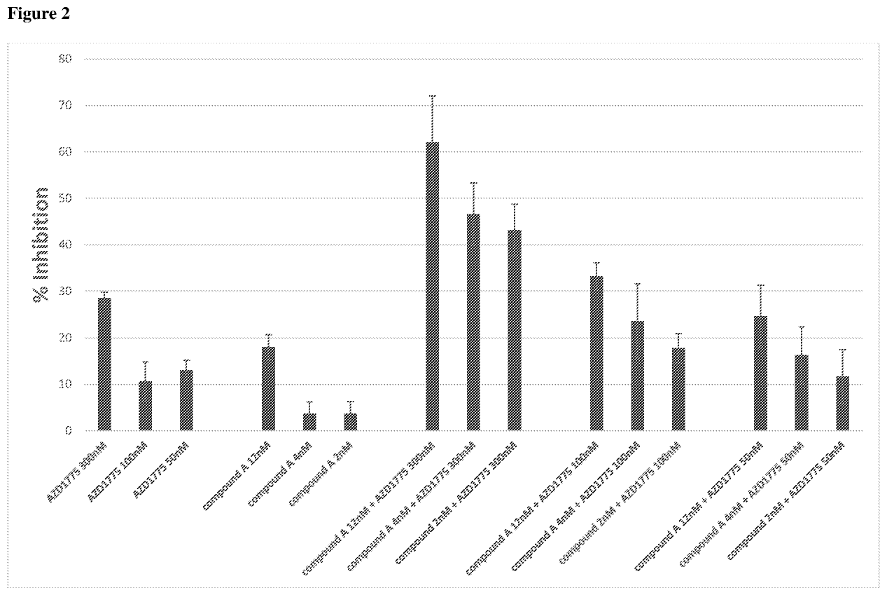

MCF-7 cells were cultured in DMEM medium with 10% Fetal bovine serum. The cells growing in an exponential growth phase were seeded at 1000 cells/well in 96 cell plates and treated with Compound A at 2 nM, 4 nM and 12 nM; Compound 1 (AZD1775) at 50, 100 and 300 nM, as single agents and in combination. After 5 days treatment, CellTiter-Glo luminescence cell viability assay (Promega) were used to measure inhibition of cell proliferation. The results are shown in FIG. 2. The results indicate that a combination of Compound (A) with Compound 1 (AZD1775) induced greater cell proliferation inhibition than each compound alone.

### Xenograft Tumor Model

MCF-7 cells were cultured in DMEM medium with 10% Fetal bovine serum. The cells growing in an exponential growth phase were seeded at 1000 cells/well in 96 cell plates and treated with Compound A at 2 nM, 4 nM and 12 nM; Compound 1 (AZD1775) at 50, 100 and 300 nM, as single agents and in combination. Plates were incubated at 37° C., 5% CO₂, after 5 days treatment, CellTiter-Glo luminescence cell viability assay (Promega) were used to measure inhibition of cell proliferation. The plates were then equilibrated to room temperature for 10 minutes and CellTiter-Glo Reagent (Promega kit) was added to each well in the plate (100 uL/well). The contents were mixed for 2 min on an orbital shaker and plates were stabilized at room temperature for 10 minutes followed by reading on a SpectraMaxR M5e (Molecular Probe) luminescence plate reader according to CellTiter-Glo protocol. Percent inhibition was calculated using the following formula: % inhibition=(RLU*100/(RLU of the cell background)). The results are shown in FIG. 2. The results indicate that a combination of Compound (A) with Compound 1 (AZD1775) induced greater cell proliferation inhibition than each compound alone.

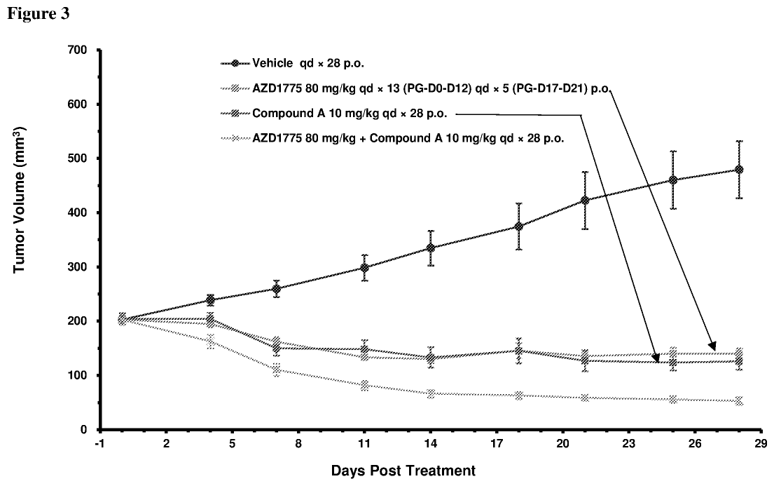

As shown in FIG. 3, Compound (A) at 10 mg/kg and Compound 1 (AZD1775) exhibited antitumor activity with TGI values of 128.3% and 122.8% respectively. Compound (A) at 10 mg/kg in combination with Compound 1 (AZD1775) at 80 mg/kg, showed significant antitumor activity with a TGI of 154.3%. The data provided herein demonstrates that a combination of a SERD inhibitor and a WEE1 inhibitor described herein can be used to treat a disease or condition described herein.

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the disclosure.

What is claimed is:

1. Use of a combination of compounds for treating a disease or condition, wherein the combination includes an effective amount of Compound (A) and an effective amount of one or more of Compound (B), or a pharmaceutically acceptable salt thereof, wherein:
the Compound (A) has the structure: 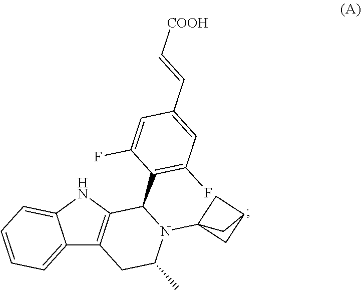 [SMILES: C[C@@H]1Cc2c([nH]c3ccccc23)[C@@H](c2c(F)cc(/C=C/C(=O)O)cc2F)N1C12CC(C1)C2]
and
the one or more of Compound (B) is a WEE1 inhibitor, or a pharmaceutically acceptable salt thereof,
wherein the WEE1 inhibitor is selected from the group consisting of 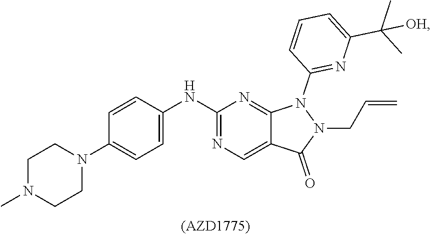 [SMILES: C=CCn1c(=O)c2cnc(Nc3ccc(N4CCN(C)CC4)cc3)nc2n1-c1cccc(C(C)(C)O)n1]
NUV-569, IMP7068, Debio 0123, 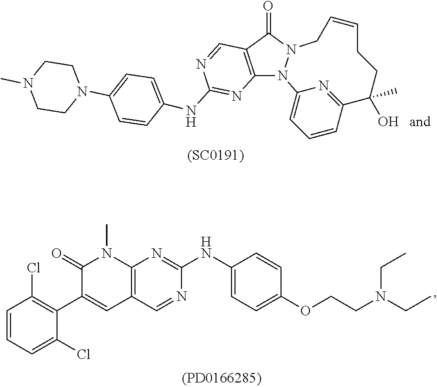
or a pharmaceutically acceptable salt of any of the foregoing.

2. Use of a combination of compounds for treating a disease or condition, wherein the combination includes an effective amount of Compound (C) and an effective amount of one or more of Compound (B), or a pharmaceutically acceptable salt thereof, wherein:
the Compound (C) has the structure: 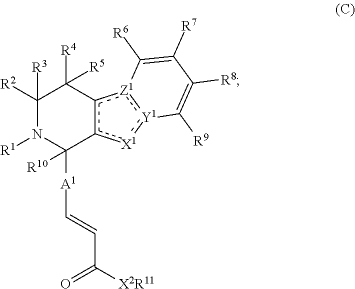
wherein:
X¹, Y¹ and Z¹ are each independently C or N;
with the first proviso that at least one of X¹, Y¹ and Z¹ is N;
with the second proviso that each of X¹, Y¹ and Z¹ is uncharged;
with third proviso that two of the dotted lines indicate double bonds;
with the fourth proviso that the valencies of X¹, Y¹ and Z¹ can be each independently satisfied by attachment to a substituent selected from H and R¹²;
X² is O;
A¹ is selected from the group consisting of an optionally substituted cycloalkyl, an optionally substituted aryl, an optionally substituted heteroaryl and an optionally substituted heterocyclyl;
R¹ is selected from the group consisting of an optionally substituted C₁₋₆ alkyl, an optionally substituted cycloalkyl, an optionally substituted cycloalkenyl, an optionally substituted aryl, an optionally substituted heteroaryl, an optionally substituted heterocyclyl, an optionally substituted cycloalkyl(C₁₋₆ alkyl), an optionally substituted cycloalkenyl(C₁₋₆ alkyl), an optionally substituted aryl(C₁₋₆ alkyl), an optionally substituted heteroaryl(C₁₋₆ alkyl) and an optionally substituted heterocyclyl(C₁₋₆ alkyl);
R² and R³ are each independently selected from the group consisting of hydrogen, halogen, an optionally substituted C₁₋₆ alkyl and an optionally substituted C₁₋₆ haloalkyl; or R² and R³ together with the carbon to which R² and R³ are attached form an optionally substituted cycloalkyl, an optionally substituted cycloalkenyl or an optionally substituted heterocyclyl;
R⁴ and R⁵ are each independently selected from the group consisting of hydrogen, halogen, an optionally substituted C₁₋₆ alkyl and an optionally substituted C₁₋₆ haloalkyl; or R⁴ and R⁵ together with the carbon to which R⁴ and R⁵ are attached form an optionally substituted cycloalkyl, an optionally substituted cycloalkenyl or an optionally substituted heterocyclyl;
R⁶, R⁷, R⁸ and R⁹ are each independently selected from the group consisting of hydrogen, halogen, hydroxy, an optionally substituted alkyl, an optionally substituted alkoxy, an optionally substituted haloalkyl, an optionally substituted mono-substituted amine, and an optionally substituted di-substituted amine;
R¹⁰ is hydrogen, halogen, an optionally substituted alkyl, or an optionally substituted cycloalkyl;
R¹¹ is hydrogen;
R¹² is hydrogen, halogen, an optionally substituted C₁₋₃ alkyl, an optionally substituted C₁₋₃ haloalkyl or an optionally substituted C₁₋₃ alkoxy; and
provided that the Compound (C) cannot be 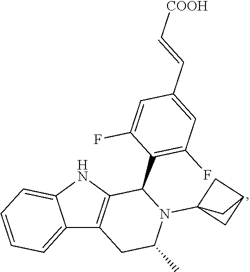 [SMILES: C[C@@H]1Cc2c([nH]c3ccccc23)[C@@H](c2c(F)cc(/C=C/C(=O)O)cc2F)N1C12CC(C1)C2]
or a pharmaceutically acceptable salt thereof; and
the one or more of Compound (B) is a WEE1 inhibitor, or a pharmaceutically acceptable salt thereof,
wherein the WEE1 inhibitor is selected from the group consisting of 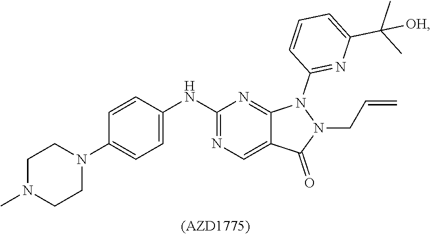 [SMILES: C=CCn1c(=O)c2cnc(Nc3ccc(N4CCN(C)CC4)cc3)nc2n1-c1cccc(C(C)(C)O)n1]
NUV-569, IMP7068, Debio 0123, 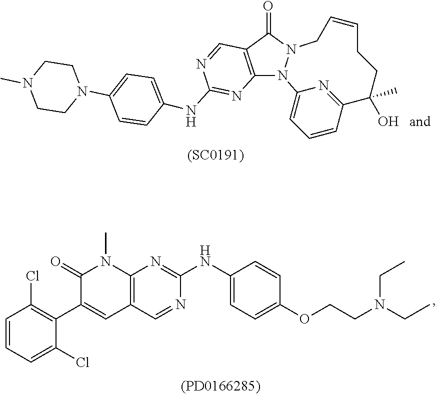
or a pharmaceutically acceptable salt of any of the foregoing

3. The use of claim 2, wherein for the Compound (C) when X¹ is NH; Y¹ and Z¹ are each C; A¹ is a phenyl, 2-fluorophenyl or 2,6-difluorophenyl; R² and R³ are each methyl or one of R² and R³ is hydrogen and the other of R² and R³ is methyl; and R⁴, R⁵, R⁶, R⁷, R⁸, R⁹ and R¹⁰ are each hydrogen; then R¹ cannot be 2-hydroxyethyl, 2-methylpropyl, 2-fluoro-2-methylpropyl, 3-fluoro-2-methylpropyl, 3-hydroxy-2-methylpropyl or 2-fluoro-3-hydroxy-2-methylpropyl.

4. The use of claim 2 or 3, wherein the Compound (C) is selected from the group consisting of: 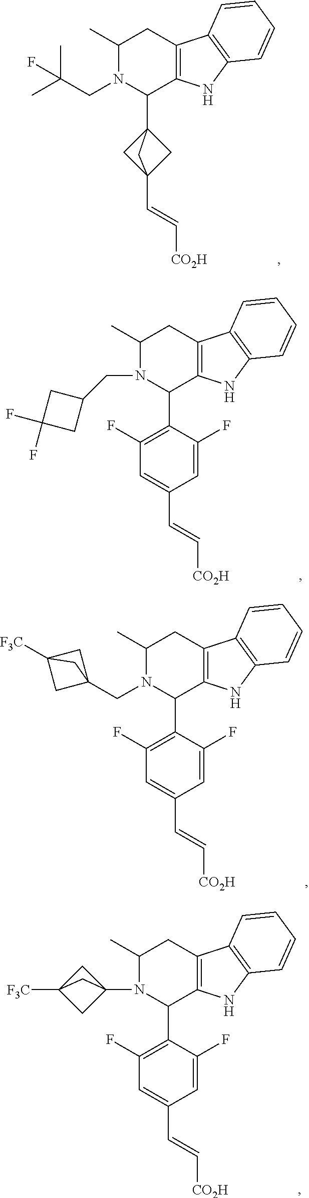 [SMILES: CC1Cc2c([nH]c3ccccc23)C(C23CC(/C=C/C(=O)O)(C2)C3)N1CC(C)(C)F ; CC1Cc2c([nH]c3ccccc23)C(c2c(F)cc(/C=C/C(=O)O)cc2F)N1CC1CC(F)(F)C1 ; CC1Cc2c([nH]c3ccccc23)C(c2c(F)cc(/C=C/C(=O)O)cc2F)N1CC12CC(C(F)(F)F)(C1)C2 ; CC1Cc2c([nH]c3ccccc23)C(c2c(F)cc(/C=C/C(=O)O)cc2F)N1C12CC(C(F)(F)F)(C1)C2] 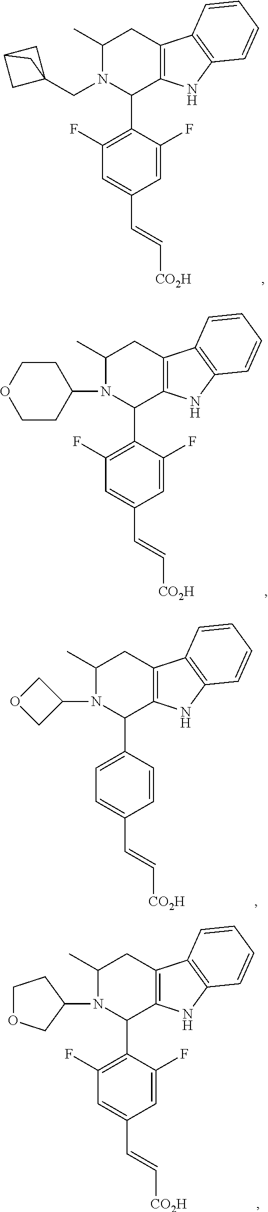 [SMILES: CC1Cc2c([nH]c3ccccc23)C(c2c(F)cc(/C=C/C(=O)O)cc2F)N1CC12CC(C1)C2 ; CC1Cc2c([nH]c3ccccc23)C(c2c(F)cc(/C=C/C(=O)O)cc2F)N1C1CCOCC1 ; CC1Cc2c([nH]c3ccccc23)C(c2ccc(/C=C/C(=O)O)cc2)N1C1COC1 ; CC1Cc2c([nH]c3ccccc23)C(c2c(F)cc(/C=C/C(=O)O)cc2F)N1C1CCOC1] 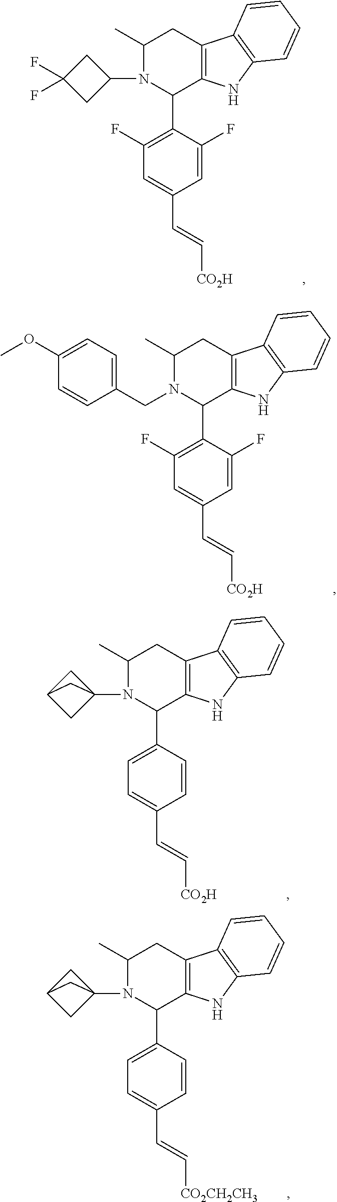 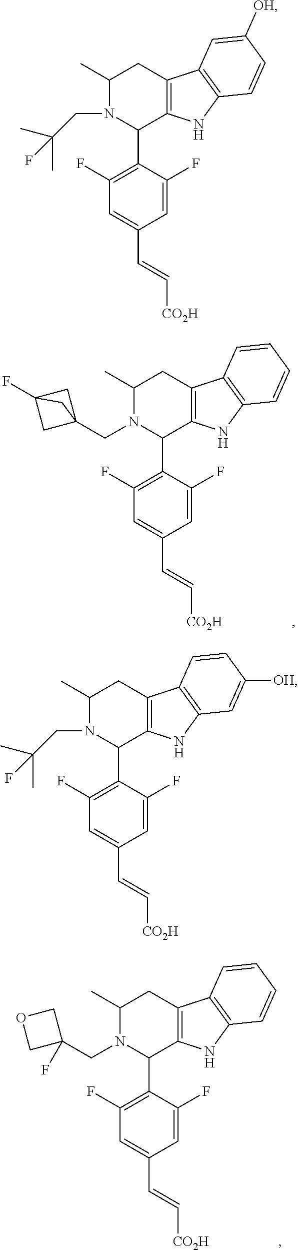 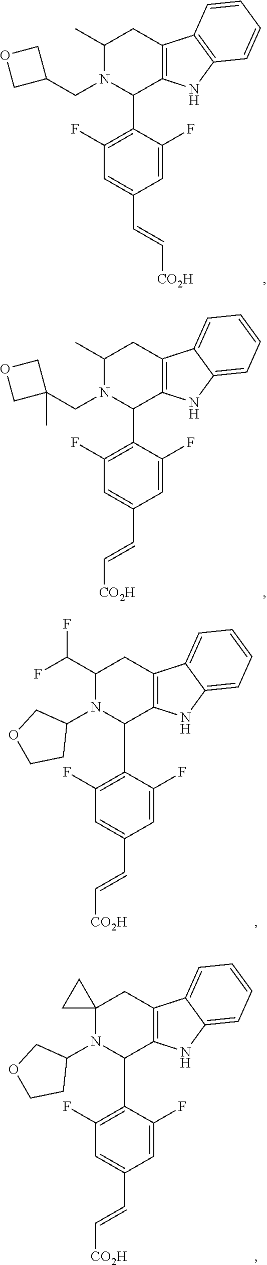 [SMILES: CC1Cc2c([nH]c3ccccc23)C(c2c(F)cc(/C=C/C(=O)O)cc2F)N1CC1COC1 ; CC1Cc2c([nH]c3ccccc23)C(c2c(F)cc(/C=C/C(=O)O)cc2F)N1CC1(C)COC1 ; O=C(O)/C=C/c1cc(F)c(C2c3[nH]c4ccccc4c3CC(C(F)F)N2C2CCOC2)c(F)c1 ; O=C(O)/C=C/c1cc(F)c(C2c3[nH]c4ccccc4c3CC3(CC3)N2C2CCOC2)c(F)c1] 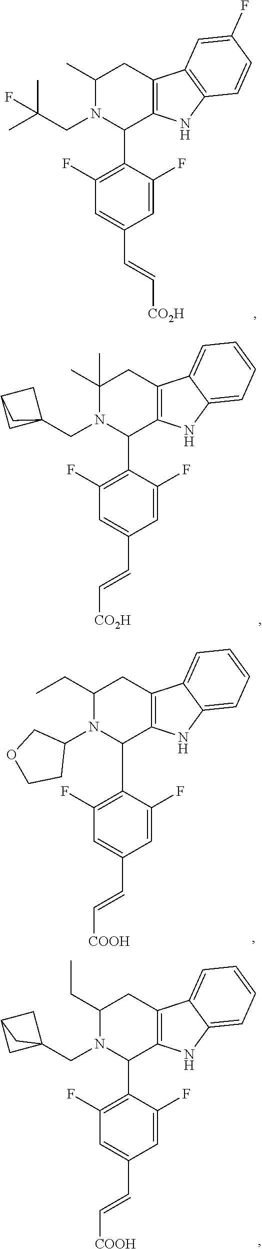 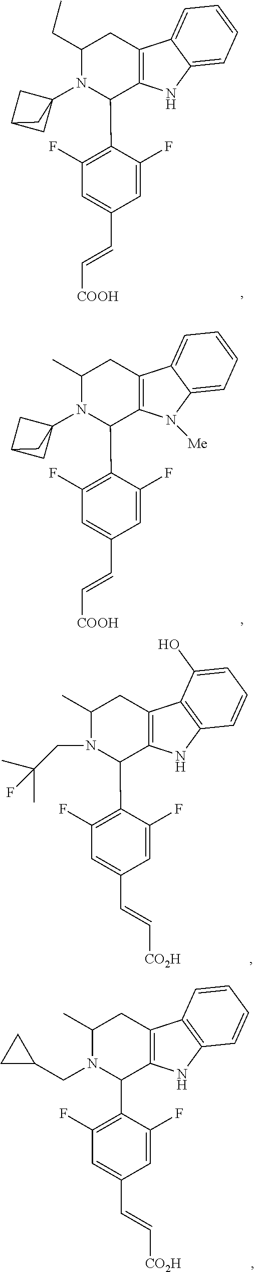 [SMILES: CCC1Cc2c([nH]c3ccccc23)C(c2c(F)cc(/C=C/C(=O)O)cc2F)N1C12CC(C1)C2 ; CC1Cc2c(n(C)c3ccccc23)C(c2c(F)cc(/C=C/C(=O)O)cc2F)N1C12CC(C1)C2 ; CC1Cc2c([nH]c3cccc(O)c23)C(c2c(F)cc(/C=C/C(=O)O)cc2F)N1CC(C)(C)F ; CC1Cc2c([nH]c3ccccc23)C(c2c(F)cc(/C=C/C(=O)O)cc2F)N1CC1CC1] 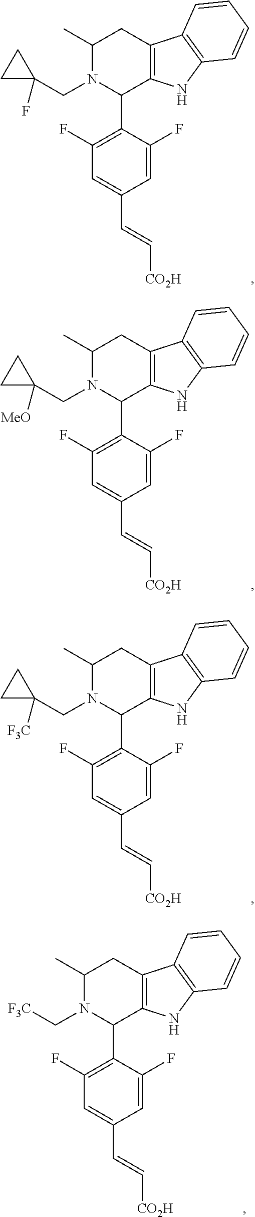 [SMILES: CC1Cc2c([nH]c3ccccc23)C(c2c(F)cc(/C=C/C(=O)O)cc2F)N1CC1(F)CC1 ; COC1(CN2C(C)Cc3c([nH]c4ccccc34)C2c2c(F)cc(/C=C/C(=O)O)cc2F)CC1 ; CC1Cc2c([nH]c3ccccc23)C(c2c(F)cc(/C=C/C(=O)O)cc2F)N1CC1(C(F)(F)F)CC1 ; CC1Cc2c([nH]c3ccccc23)C(c2c(F)cc(/C=C/C(=O)O)cc2F)N1CC(F)(F)F] 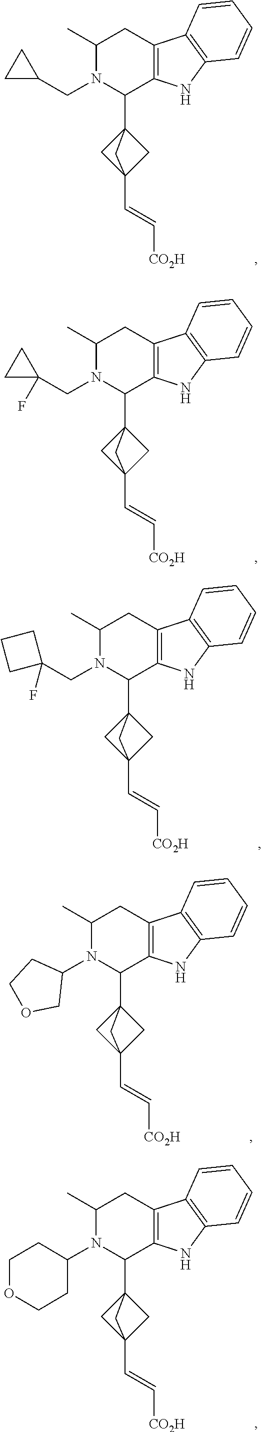 [SMILES: CC1Cc2c([nH]c3ccccc23)C(C23CC(/C=C/C(=O)O)(C2)C3)N1CC1CC1 ; CC1Cc2c([nH]c3ccccc23)C(C23CC(/C=C/C(=O)O)(C2)C3)N1CC1(F)CC1 ; CC1Cc2c([nH]c3ccccc23)C(C23CC(/C=C/C(=O)O)(C2)C3)N1CC1(F)CCC1 ; CC1Cc2c([nH]c3ccccc23)C(C23CC(/C=C/C(=O)O)(C2)C3)N1C1CCOC1 ; CC1Cc2c([nH]c3ccccc23)C(C23CC(/C=C/C(=O)O)(C2)C3)N1C1CCOCC1] 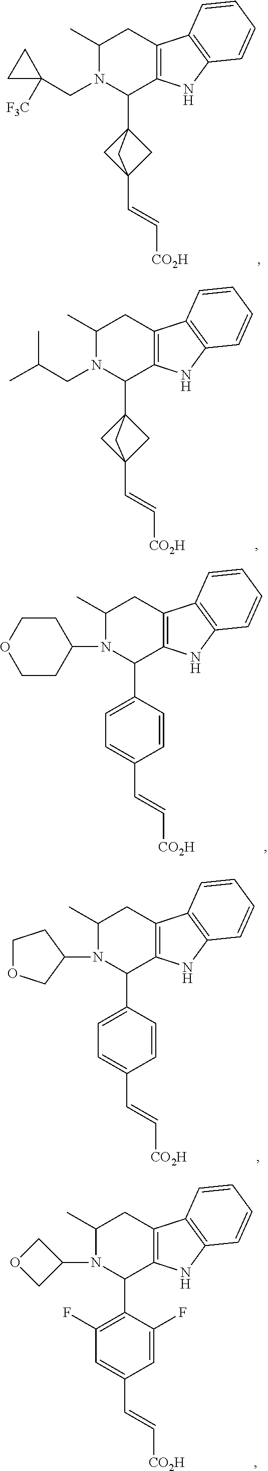 [SMILES: CC1Cc2c([nH]c3ccccc23)C(C23CC(/C=C/C(=O)O)(C2)C3)N1CC1(C(F)(F)F)CC1 ; CC(C)CN1C(C)Cc2c([nH]c3ccccc23)C1C12CC(/C=C/C(=O)O)(C1)C2 ; CC1Cc2c([nH]c3ccccc23)C(c2ccc(/C=C/C(=O)O)cc2)N1C1CCOCC1 ; CC1Cc2c([nH]c3ccccc23)C(c2ccc(/C=C/C(=O)O)cc2)N1C1CCOC1 ; CC1Cc2c([nH]c3ccccc23)C(c2c(F)cc(/C=C/C(=O)O)cc2F)N1C1COC1] 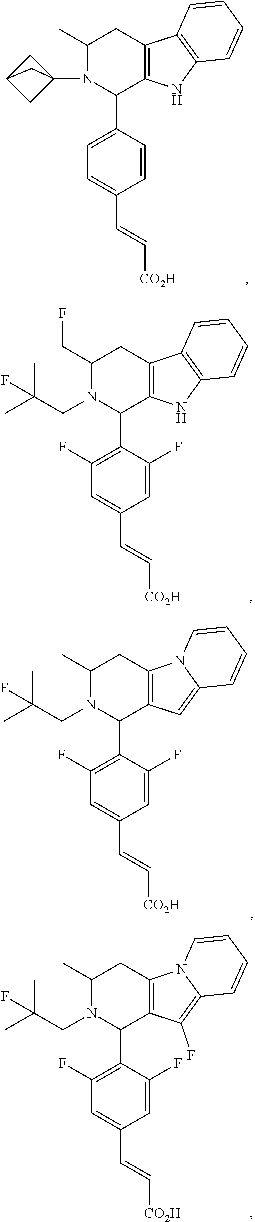 [SMILES: CC1Cc2c([nH]c3ccccc23)C(c2ccc(/C=C/C(=O)O)cc2)N1C12CC(C1)C2 ; CC(C)(F)CN1C(CF)Cc2c([nH]c3ccccc23)C1c1c(F)cc(/C=C/C(=O)O)cc1F ; CC1Cc2c(cc3ccccn23)C(c2c(F)cc(/C=C/C(=O)O)cc2F)N1CC(C)(C)F ; CC1Cc2c(c(F)c3ccccn23)C(c2c(F)cc(/C=C/C(=O)O)cc2F)N1CC(C)(C)F] 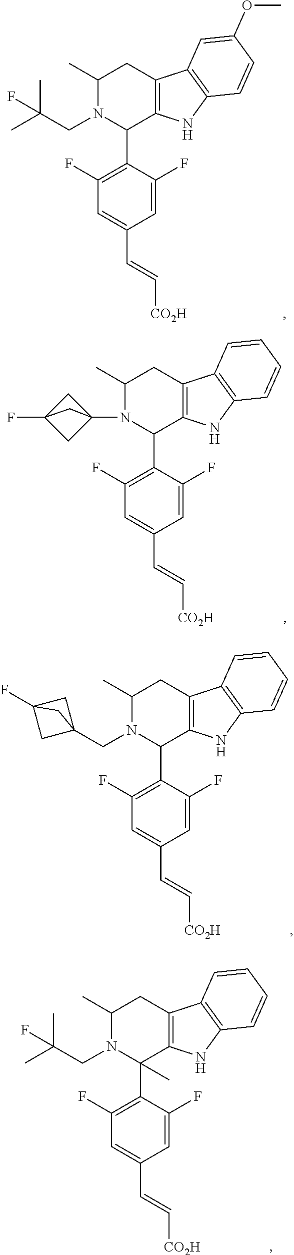 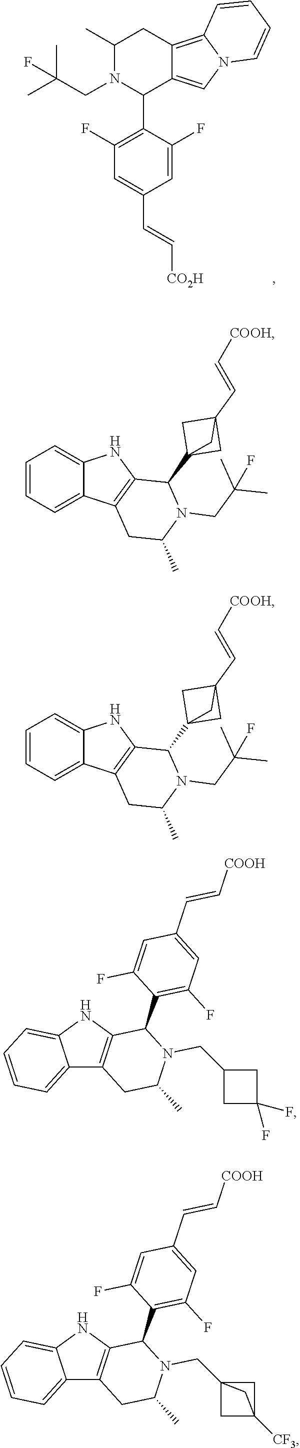 [SMILES: CC1Cc2c(cn3ccccc23)C(c2c(F)cc(/C=C/C(=O)O)cc2F)N1CC(C)(C)F ; C[C@@H]1Cc2c([nH]c3ccccc23)[C@@H](C23CC(/C=C/C(=O)O)(C2)C3)N1CC(C)(C)F ; C[C@@H]1Cc2c([nH]c3ccccc23)[C@H](C23CC(/C=C/C(=O)O)(C2)C3)N1CC(C)(C)F ; C[C@@H]1Cc2c([nH]c3ccccc23)[C@@H](c2c(F)cc(/C=C/C(=O)O)cc2F)N1CC1CC(F)(F)C1 ; C[C@@H]1Cc2c([nH]c3ccccc23)[C@@H](c2c(F)cc(/C=C/C(=O)O)cc2F)N1CC12CC(C(F)(F)F)(C1)C2] 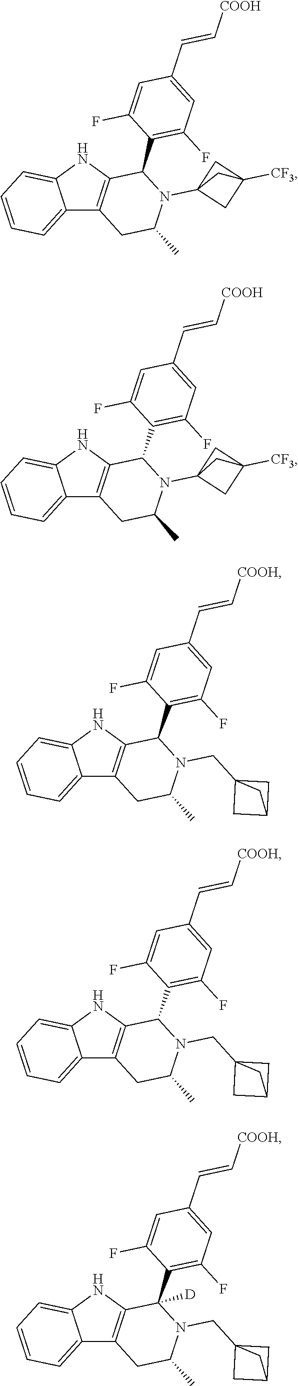 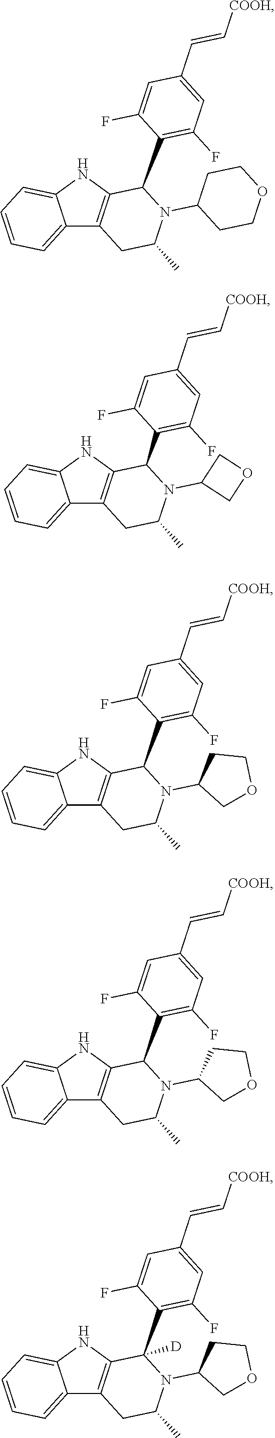 [SMILES: C[C@@H]1Cc2c([nH]c3ccccc23)[C@@H](c2c(F)cc(/C=C/C(=O)O)cc2F)N1C1CCOCC1 ; C[C@@H]1Cc2c([nH]c3ccccc23)[C@@H](c2c(F)cc(/C=C/C(=O)O)cc2F)N1C1COC1 ; C[C@@H]1Cc2c([nH]c3ccccc23)[C@@H](c2c(F)cc(/C=C/C(=O)O)cc2F)N1[C@H]1CCOC1 ; C[C@@H]1Cc2c([nH]c3ccccc23)[C@@H](c2c(F)cc(/C=C/C(=O)O)cc2F)N1[C@@H]1CCOC1 ; [2H][C@@]1(c2c(F)cc(/C=C/C(=O)O)cc2F)c2[nH]c3ccccc3c2C[C@@H](C)N1[C@H]1CCOC1] 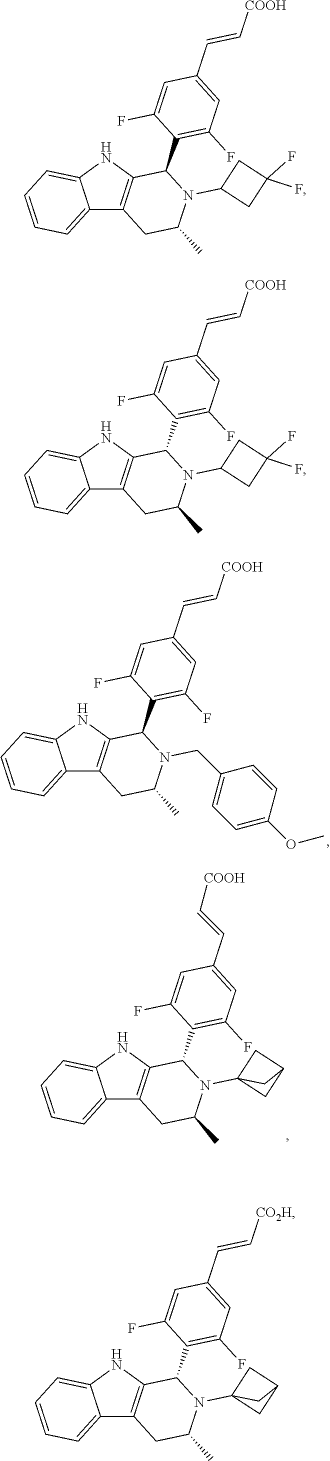 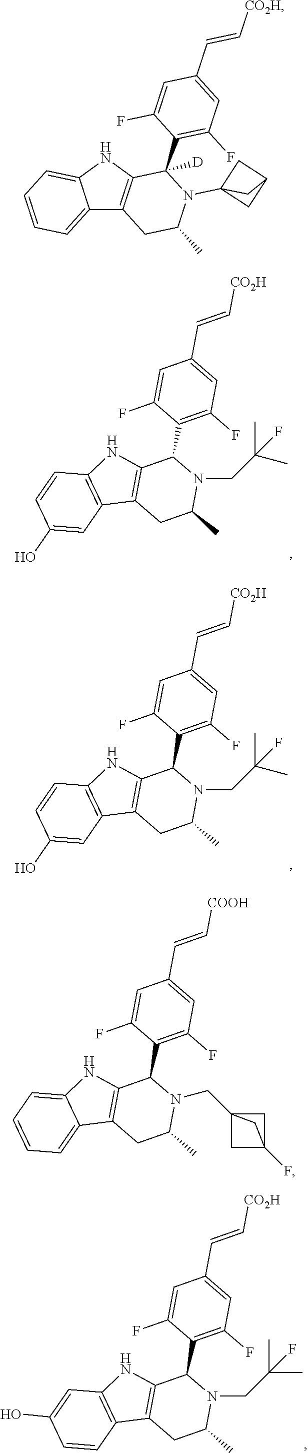 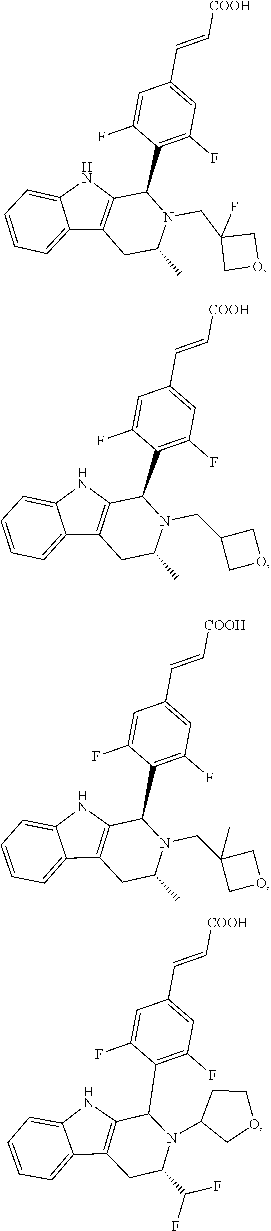 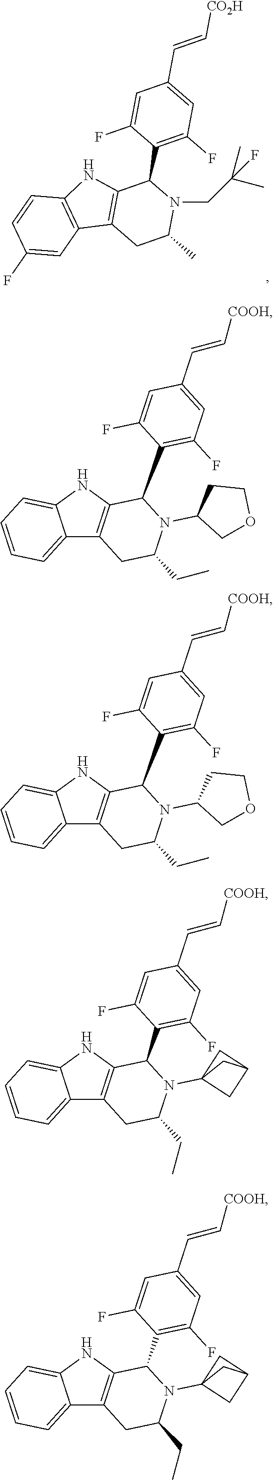 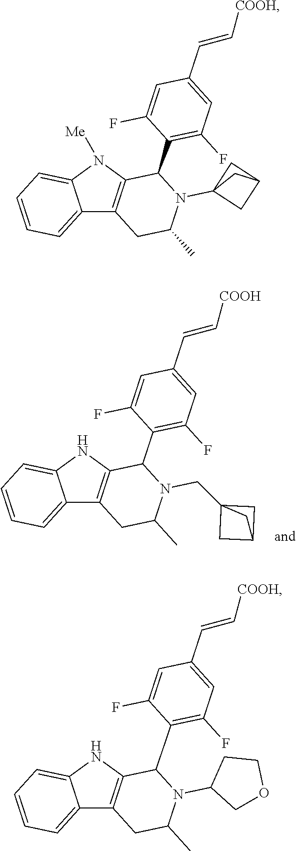 [SMILES: C[C@@H]1Cc2c(n(C)c3ccccc23)[C@@H](c2c(F)cc(/C=C/C(=O)O)cc2F)N1C12CC(C1)C2 ; CC1Cc2c([nH]c3ccccc23)C(c2c(F)cc(/C=C/C(=O)O)cc2F)N1CC12CC(C1)C2 ; CC1Cc2c([nH]c3ccccc23)C(c2c(F)cc(/C=C/C(=O)O)cc2F)N1C1CCOC1]
or a pharmaceutically acceptable salt of any of the foregoing.

5. The use of any one of claims 1-4, wherein the disease or condition is selected from the group consisting of a breast cancer, a cervical cancer, an ovarian cancer, an uterine cancer, a vaginal cancer, a vulvar cancer, a brain cancer, a cervicocerebral cancer, an esophageal cancer, a thyroid cancer, a small cell cancer, a non-small cell cancer, a lung cancer, a stomach cancer, a gallbladder/bile duct cancer, a liver cancer, a pancreatic cancer, a colon cancer, a rectal cancer, a choriocarcinoma, an uterus body cancer, an uterocervical cancer, a renal pelvis/ureter cancer, a bladder cancer, a prostate cancer, a penis cancer, a testicular cancer, a fetal cancer, a Wilms' cancer, a skin cancer, a malignant melanoma, a neuroblastoma, an osteosarcoma, an Ewing's tumor, a soft part sarcoma, an acute leukemia, a chronic lymphatic leukemia, a chronic myelocytic leukemia, polycythemia vera, a malignant lymphoma, multiple myeloma, a Hodgkin's lymphoma, and a non-Hodgkin's lymphoma.

6. The use of any one of claims 1-4, wherein the disease or condition is a breast cancer, a cervical cancer, an ovarian cancer, an uterine cancer, a vaginal cancer, and a vulvar cancer.

7. The use of claim 6, wherein the disease or condition is a breast cancer.

8. The use of any one of claims 5-7, wherein the breast cancer that does not include any point mutations ER mutations.

9. The use of any one of claims 5-7, wherein the disease or condition is breast cancer that has at least one point mutation within the Estrogen Receptor 1 (ESR1) that encodes Estrogen receptor alpha (ERα), wherein the mutation is selected from the group consisting of: K303R, D538G, Y537S, E380Q, Y537C, Y537N, A283V, A546D, A546T, A58T, A593D, A65V, C530L, D411H, E279V, E471D, E471V, E523Q, E542G, F461V, F97L, G145D, G160D, G274R, G344D, G420D, G442R, G557R, H524L, K252N, K481N, K531E, L370F, L453F, L466Q, L497R, L536H, L536P, L536Q, L536R, L540Q, L549P, M388L, M396V, M421V, M437I, M522I, N156T, N532K, N69K, P147Q, P222S, P535H, R233G, R477Q, R503W, R555H, S282C, S329Y, S338G, S432L, S463P, S47T, S576L, V392I, V418E, V478L, V533M, V534E, Y537D and Y537H.

10. The use of any one of claims 5-9, wherein the breast cancer is ER positive breast cancer.

11. The use of any one of claims 5-9, wherein the breast cancer is ER positive/HER2-negative breast cancer.

12. The use of any one of claims 5-11, wherein the breast cancer is local breast cancer.

13. The use of any one of claims 5-11, wherein the breast cancer is metastatic breast cancer.

14. The use of any one of claims 5-13, wherein the breast cancer is recurrent breast cancer.

15. The use of any one of claims 5-14, wherein the breast cancer has been previously treated with an endocrine therapy.

16. The use of claim 15, wherein the treatment was with a selective ER modulator (SERM).

17. The use of claim 16, wherein the selective ER modulator is selected from the group consisting of tamoxifen, raloxifene, ospemifene, bazedoxifene, toremifene and lasofoxifene, or a pharmaceutically acceptable salt of any of the foregoing.

18. The use of claim 15, wherein the treatment was with a selective ER degrader (SERD).

19. The use of claim 18, wherein the selective ER degrader is selected from the group consisting of fulvestrant, (E)-3-[3,5-Difluoro-4-[(R1R,3R)-2-(2-fluoro-2-methylpropyl)-3-methyl-1,3,4,9-tetrahydropyrido[3,4-b]indol-1-yl]phenyl]prop-2-enoic acid (AZD9496) (R)-6-(2-(ethyl(4-(2-(ethylamino)ethyl)benzyl)amino)-4-methoxyphenyl)-5,6,7,8-tetrahydronaphthalen-2-ol (elacestrant, RAD1901), (E)-3-(4-((E)-2-(2-chloro-4-fluorophenyl)-1-(1H-indazol-5-yl)but-1-en-1-yl)phenyl)acrylic acid (Brilanestrant, ARN-810, GDC-0810), (E)-3-(4-((2-(2-(1,1-difluoroethyl)-4-fluorophenyl)-6-hydroxybenzo[b]thiophen-3-yl)oxy)phenyl)acrylic acid (LSZ102), (E)-N,N-dimethyl-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide (H3B-6545), (E)-3-(4-((2-(4-fluoro-2,6-dimethylbenzoyl)-6-hydroxybenzo[b]thiophen-3-yl)oxy)phenyl)acrylic acid (rintodestrant, G1T48), D-0502, SHR9549, ARV-471, 3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoropropan-1-ol (giredestrant, GDC-9545), (S)-8-(2,4-dichlorophenyl)-9-(4-((1-(3-fluoropropyl)pyrrolidin-3-yl)oxy)phenyl)-6,7-dihydro-5H-benzo[7]annulene-3-carboxylic acid (SAR439859), N-[1-(3-fluoropropyl)azetidin-3-yl]-6-[(R6S,8R)-8-methyl-7-(2,2,2-trifluoroethyl)-6,7,8,9-tetrahydro-3H-pyrazolo[4,3-f]isoquinolin-6-yl]pyridin-3-amine (AZD9833), OP-1250 and LY3484356, or a pharmaceutically acceptable salt of any of the foregoing.

20. The use of claim 15, wherein the treatment was with an aromatase inhibitor.

21. The use of claim 20, wherein the aromatase inhibitor is a steroidal aromatase inhibitor.

22. The use of claim 21, wherein the steroidal aromatase inhibitor is selected from the group consisting of exemestane and testolactone, or a pharmaceutically acceptable salt of any of the foregoing.

23. The use of claim 20, wherein the aromatase inhibitor is a non-steroidal aromatase inhibitor.

24. The use of claim 23, wherein the non-steroidal aromatase inhibitor is selected from the group consisting of anastazole and letrazole, or a pharmaceutically acceptable salt of any of the foregoing.

25. The use of any one of claims 5-13, wherein the breast cancer has not been previously treated.

26. The use of any one of claim 5-25, wherein the breast cancer is present in a woman.

27. The use of claim 26, wherein the subject is a premenopausal woman.

28. The use of claim 26, wherein the subject is a perimenopausal woman.

29. The use of claim 26, wherein the subject is a menopausal woman.

30. The use of claim 26, wherein the breast cancer is present in a postmenopausal woman.

31. The use of any one of claim 5-25, wherein the breast cancer is present a man.

32. The use of any one of claim 5-31, wherein the breast cancer is present in a subject that has a serum estradiol level in the range of >15 pg/mL to 350 pg/mL.

33. The use of any one of claim 5-31, wherein the breast cancer is present in a subject that has a serum estradiol level≤15 pg/mL.

34. The use of any one of claim 5-31, wherein the breast cancer is present in a subject that has a serum estradiol level≤10 pg/mL.
